(19) United States
(12) Patent Application Publication
(10) Pub. No.: US 2021/0074930 A1
(43) Pub. Date: Mar. 11, 2021

(54) SYNTHESIS OF FT4-BASED ORGANIC SEMICONDUCTING SMALL MOLECULES BY PD-CATALYZED DIRECT (HETERO)ARYLATION OR DIRECT ALKENYLATION

(71) Applicants: Corning Incorporated, Corning, NY (US); Liaoning Shihua University, Fushun (CN)

(72) Inventors: Mingqian He, Horseheads, NY (US); Yang Li, Shanghai (CN); Jing Sun, Fushun (CN); Hongxiang Wang, Shanghai (CN); Mong-dong Zhou, Fushun (CN)

(21) Appl. No.: 17/012,736

(22) Filed: Sep. 4, 2020

(30) Foreign Application Priority Data
Sep. 6, 2019 (CN) 201910842295.X

(51) Int. Cl. H01L 51/00; C09K 11/06; C07D 495/22; C08G 61/12

(52) U.S. Cl. CPC H01L 51/0074; C09K 11/06; C07D 495/22; H01L 51/0068; H01L 51/0558; H01L 51/0036; H01L 51/0043; C09K 2211/1018; C08G 61/126

(57) ABSTRACT

A method for forming organic semiconducting materials, including: providing a mixture having: a tetrathienoacene (FT4)-based monomer, and one of a thiophene-containing compound or an alkene-containing compound; and reacting the FT4-based monomer with the thiophene-containing compound or the alkene-containing compound in a one-step direct arylation reaction mechanism to form a final FT4-based organic semiconducting compound.

### BACKGROUND

### 1. Field

This application claims the benefit of priority under 35 U.S.C. § 119 of Chinese Patent Application Serial No. 201910842295.X, filed on Sep. 6, 2019, the content of which is relied upon and incorporated herein by reference in its entirety.

The disclosure relates to synthesis of tetrathienoacene (FT4)-based organic semiconducting small molecules by Pd-catalyzed direct (hetero)arylation for organic thin-film transistors (OTFTs).

### 2. Technical Background

Organic thin-film transistors (OTFTs) have garnered extensive attention as alternatives to conventional silicon-based technologies, which require high temperature and high vacuum deposition processes, as well as complex photolithographic patterning methods. Semiconducting (i.e., organic semiconductor, OSC) layers are one important component of OTFTs which can effectively influence the performance of devices.

Synthesis of tetrathienoacene (FT4)-based organic semiconducting small molecules as materials for OSC layers have been typically conducted using carbon-carbon (C—C) coupling reactions of aryl halides with organometallic aryl species by transition metal catalysts (e.g., Suzuki, Stille, Negishi and Kumada reactions). However, these techniques require extra synthesis steps and unstable compounds due to the installation of necessary organometallic moieties such as —SnR₃ for Stille-coupling, —B(OR)₃ for Suzuki-coupling, —ZnR for Negishi-coupling, and —MgX for Kumada-coupling. Alternatively, two unsubstituted arenes may undergo C—H activation and be joined by an oxidative coupling process; this requires a directing group to activate the relatively inert bonds and is unselective.

This disclosure presents improved synthesis of FT4-based organic semiconducting small molecules by Pd-catalyzed direct (hetero)arylation for OSC layers of organic thin-film transistors.

### SUMMARY

In some embodiments, a method for forming organic semiconducting materials, comprises: providing a mixture including: a tetrathienoacene (FT4)-based monomer, and one of a thiophene-containing compound or an alkene-containing compound; and reacting the FT4-based monomer with the thiophene-containing compound or the alkene-containing compound in a one-step direct arylation reaction mechanism to form a final FT4-based organic semiconducting compound.

In one aspect, which is combinable with any of the other aspects or embodiments, the reaction proceeds to a yield of at least 10% completion to form the final FT4-based organic semiconducting compound.

In one aspect, which is combinable with any of the other aspects or embodiments, the reaction proceeds to a yield of at least 20% completion to form the final FT4-based organic semiconducting compound.

In one aspect, which is combinable with any of the other aspects or embodiments, the reaction proceeds to a yield of at least 30% completion to form the final FT4-based organic semiconducting compound.

In one aspect, which is combinable with any of the other aspects or embodiments, the reaction proceeds to a yield of at least 50% completion to form the final FT4-based organic semiconducting compound.

In one aspect, which is combinable with any of the other aspects or embodiments, the final FT4-based organic semiconducting compound is an oligomer comprising at least two repeat units and at most ten repeat units.

In one aspect, which is combinable with any of the other aspects or embodiments, the final FT4-based organic semiconducting compound has a molecular weight in a range of 1000 Da to 12500 Da.

In one aspect, which is combinable with any of the other aspects or embodiments, the step of reacting is conducted in the presence of a Pd catalyst.

In one aspect, which is combinable with any of the other aspects or embodiments, the Pd catalyst comprises at least one of: Pd(OAc)₂, PdCl₂, Pd(O₂CCF₃)₂, C₈H₁₂B₂F₈N₄Pd, Pd(PPh₃)₄, Pd/C, Pd₂(dba)₃, PPh₃, P-(o-MeOPh)₃/Pd₂(dba)₃, PdCO₂(CF₃)₂, tetrakis (acetonitrile) palladium (II) tetrafluoroborate, PdCl₂(MeCN)₂, Pd₂(dba)₃.CHCl₃, Herrmann-Beller catalyst, or combinations thereof.

In one aspect, which is combinable with any of the other aspects or embodiments, the step of reacting is conducted in a solvent selected from: dimethylacetamide (DMAc), toluene, tetrahydrofuran (THF), dimethylformamide (DMF), benzotrifluoride, hexafluoroisopropanol (HFIP), 1,2-dichloroethane (DCE), dimethoxyethane (DME), hexafluorobenzene, 1,4-dioxane, mesitylene, chlorobenzene, p-xylene, o-dichlorobenzene, 1,2,4-trichlorobenzene, 1-chloronaphthalene, or combinations thereof.

In one aspect, which is combinable with any of the other aspects or embodiments, the final FT4-based organic semiconducting compound has a fluorescence intensity of at least 200 a.u., as measured 200 nm to 750 nm.

In one aspect, which is combinable with any of the other aspects or embodiments, the mixture further includes at least one of an additive, an oxidant, a base, or a ligand.

In some embodiments, an organic semiconducting material is selected from the group consisting of:

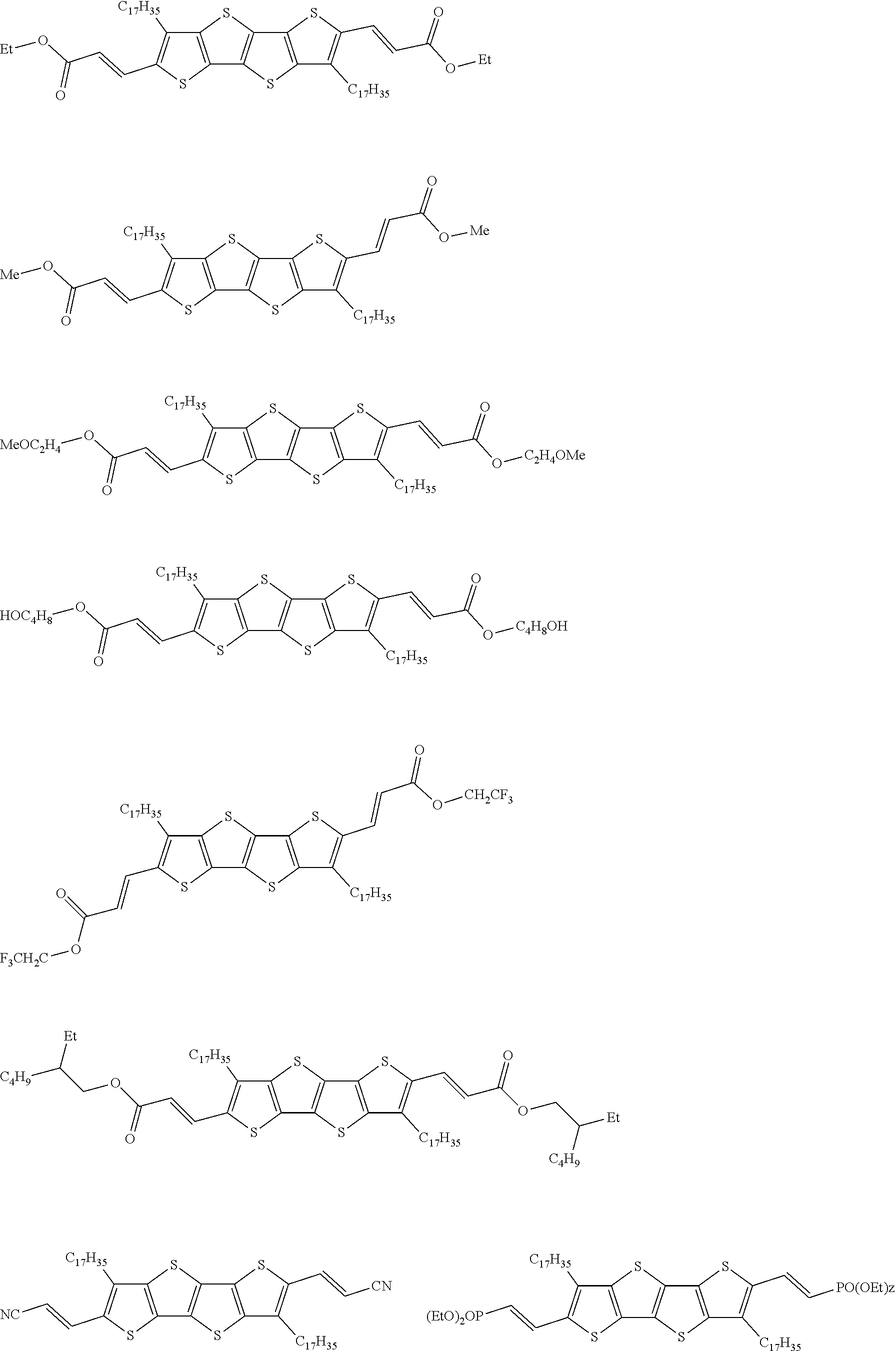

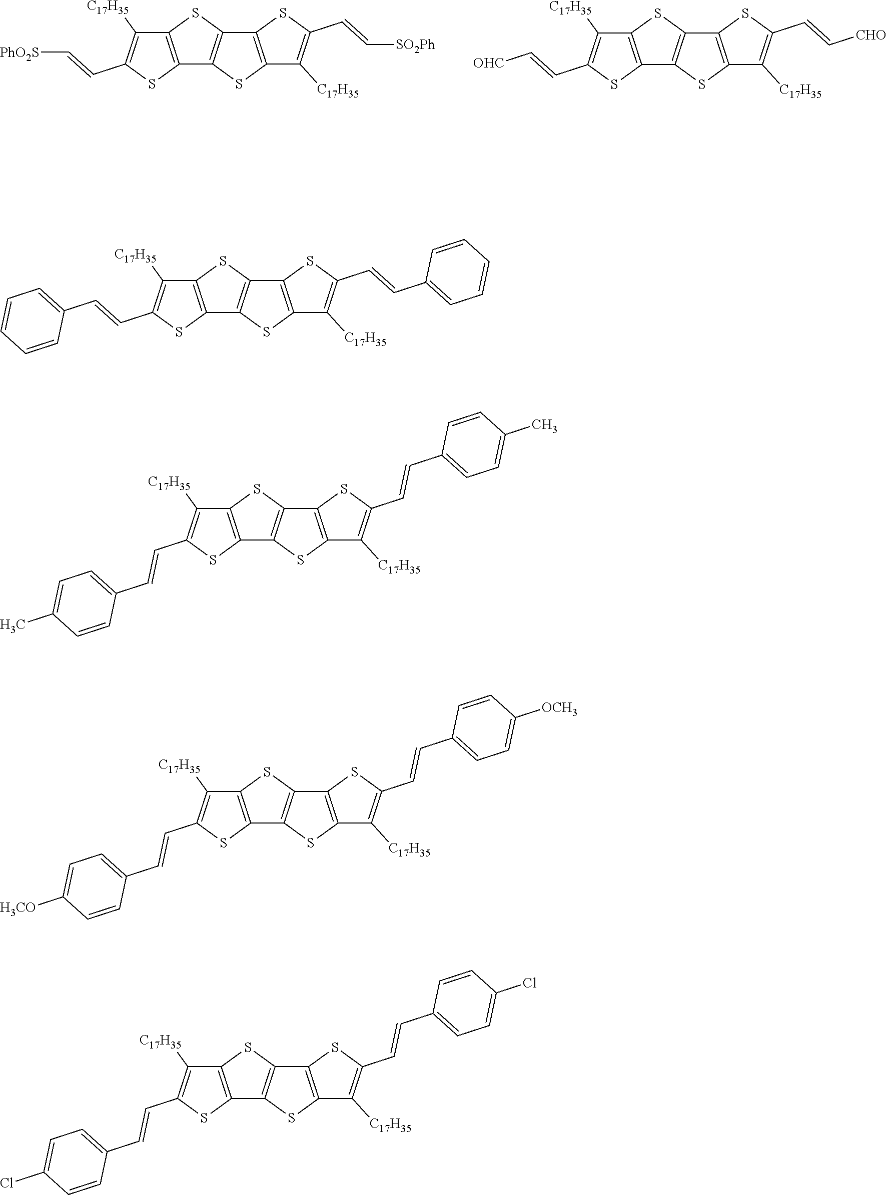

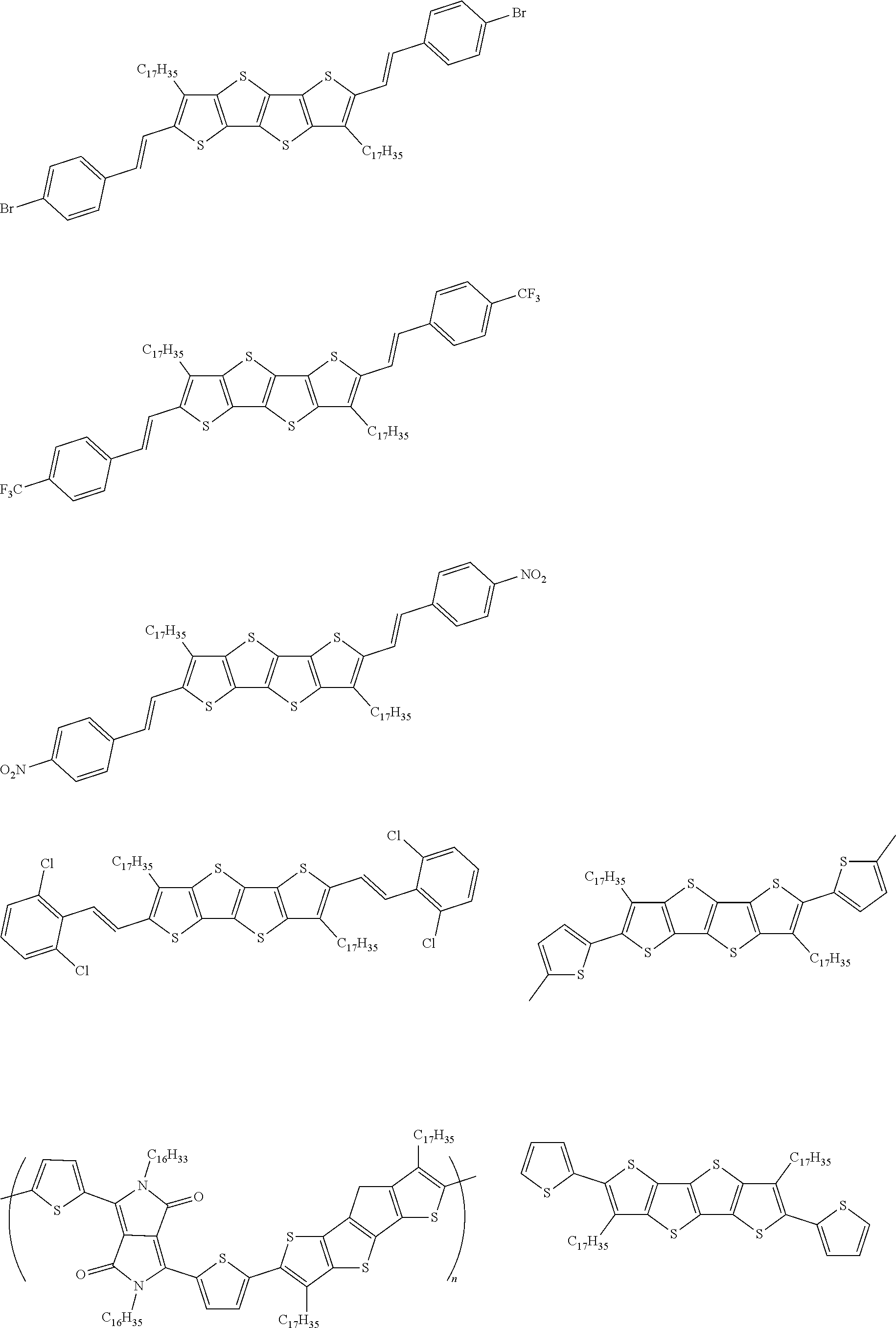

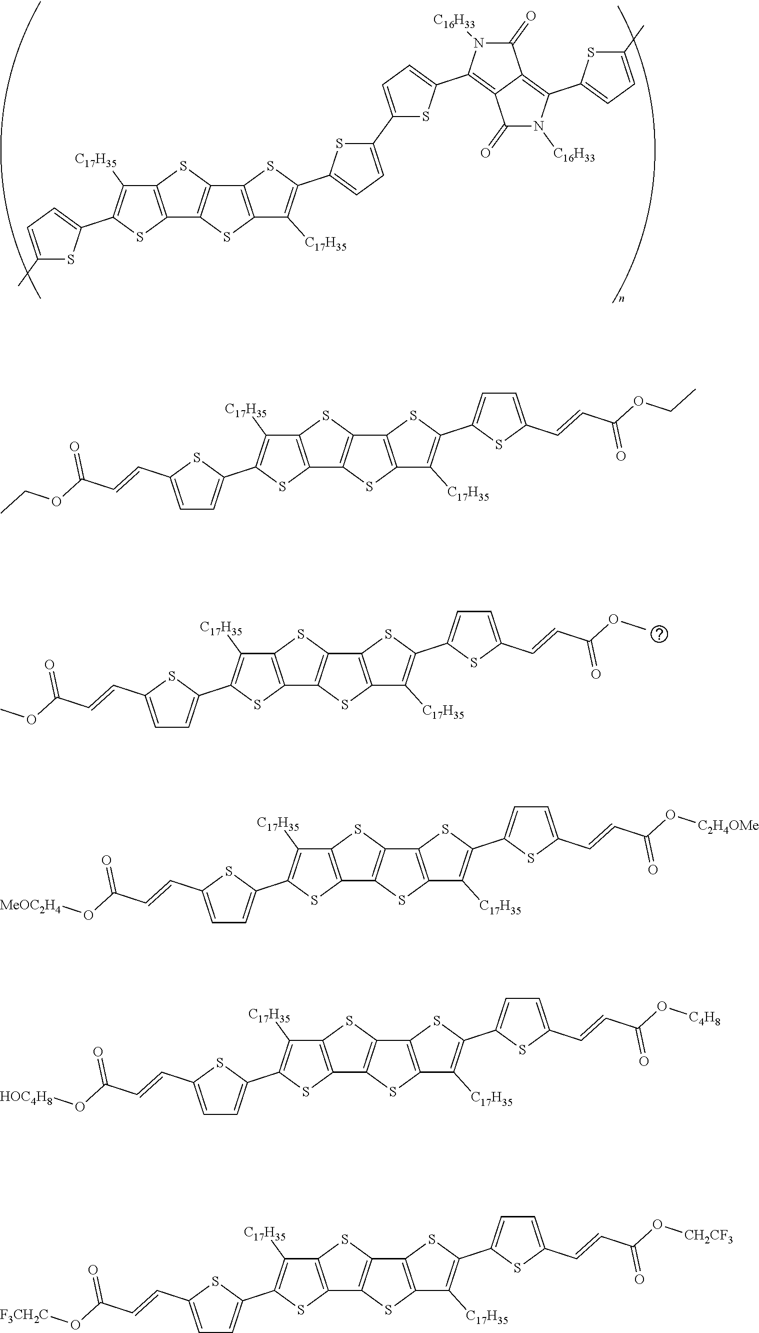

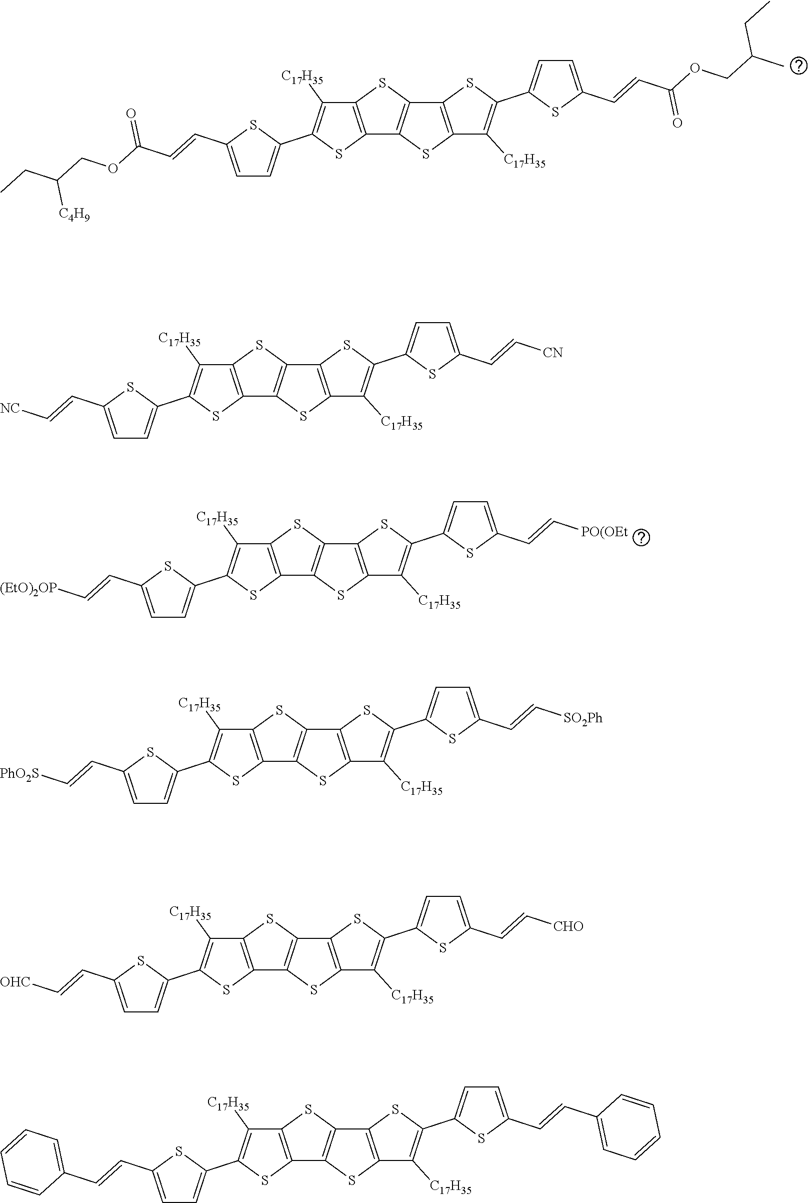

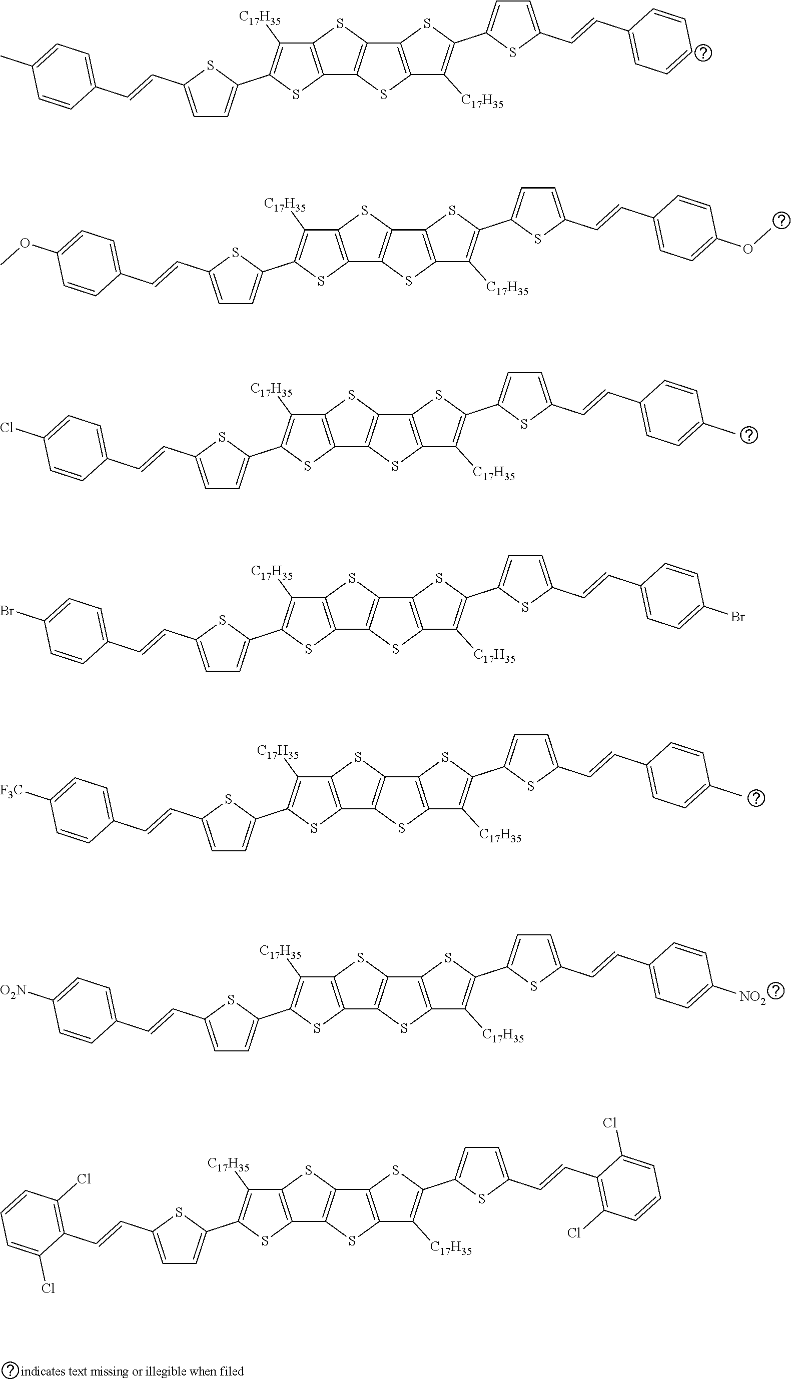

where n=2, 3, 4, or 5.

### BRIEF DESCRIPTION OF THE DRAWINGS

The disclosure will become more fully understood from the following detailed description, taken in conjunction with the accompanying figures, in which:

FIG. 1 illustrates a Pd-catalyzed aryl cross-coupling reaction, according to some embodiments.

FIG. 2 illustrates a catalytic cycle for direct (hetero)arylation between thiophene and bromobenzene with a carboxylate additive, according to some embodiments.

FIG. 3 illustrates a catalytic cycle for direct (hetero)arylation between thiophene and bromobenzene without a carboxylate additive, according to some embodiments.

FIG. 4 illustrates a mechanism of direct alkenylation with a silver (Ag) oxidant, according to some embodiments.

FIG. 5 illustrates a proton nuclear magnetic resonance (′H-NMR) spectrum of product 3a, according to some embodiments.

FIG. 6 illustrates a carbon-13 nuclear magnetic resonance (¹³C-NMR) spectrum of product 3a, according to some embodiments.

FIG. 7 illustrates an ultraviolet-visible (UV-Vis) absorption spectrum of product 3k (10⁻⁵ mol/L in dichloromethane, ethyl alcohol, and chloroform), according to some embodiments.

FIG. 8 illustrates an ultraviolet-visible (UV-Vis) absorption spectrum of product 3a (10⁻⁵ mol/L in dichloromethane), according to some embodiments.

FIG. 9 illustrates a plot of fluorescence intensity of product 3a (10⁻⁵ mol/L in dichloromethane), according to some embodiments.

FIG. 10 illustrates an exemplary OTFT device, according to some embodiments.

FIG. 11 illustrates an exemplary OTFT device, according to some embodiments.

### DETAILED DESCRIPTION

Reference will now be made in detail to exemplary embodiments which are illustrated in the accompanying drawings. Whenever possible, the same reference numerals will be used throughout the drawings to refer to the same or like parts. The components in the drawings are not necessarily to scale, emphasis instead being placed upon illustrating the principles of the exemplary embodiments. It should be understood that the present application is not limited to the details or methodology set forth in the description or illustrated in the figures. It should also be understood that the terminology is for the purpose of description only and should not be regarded as limiting.

Additionally, any examples set forth in this specification are illustrative, but not limiting, and merely set forth some of the many possible embodiments of the claimed invention. Other suitable modifications and adaptations of the variety of conditions and parameters normally encountered in the field, and which would be apparent to those skilled in the art, are within the spirit and scope of the disclosure.

### Definitions

The term “alkyl group” refers to a monoradical branched or unbranched saturated hydrocarbon chain having 1 to 40 carbon atoms. This term is exemplified by groups such as methyl, ethyl, n-propyl, isopropyl, n-butyl, isobutyl, t-butyl, pentyl, n-hexyl, n-heptyl, n-octyl, n-decyl, or tetradecyl, and the like. The alkyl group can be substituted or unsubstituted.

The term “substituted alkyl group” refers to: (1) an alkyl group as defined above, having 1, 2, 3, 4 or 5 substituents, typically 1 to 3 substituents, selected from the group consisting of alkenyl, alkynyl, alkoxy, aralkyl, aldehyde, cycloalkyl, cycloalkenyl, acyl, acylamino, acyl halide, acyloxy, amino, aminocarbonyl, alkoxycarbonylamino, azido, cyano, halogen, hydroxy, keto, thiocarbonyl, carboxy, carboxyalkyl, arylthiol, ester, heteroarylthio, heterocyclylthio, hydroxyl, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, —SO₂-aryl and —SO₂-heteroaryl, thioalkyl, vinyl ether. Unless otherwise constrained by the definition, all substituents may optionally be further substituted by 1, 2, or 3 substituents chosen from alkyl, carboxy, carboxyalkyl, aminocarbonyl, hydroxy, alkoxy, halogen, CF₃, amino, substituted amino, cyano, and —S(O)ₙR_{SO}, where R_{SO} is alkyl, aryl, or heteroaryl and n is 0, 1 or 2; or (2) an alkyl group as defined above that is interrupted by 1-10 atoms independently chosen from oxygen, sulfur and NRₐ, where Rₐ is chosen from hydrogen, alkyl, cycloalkyl, alkenyl, cycloalkenyl, alkynyl, aryl, heteroaryl and heterocyclyl. All substituents may be optionally further substituted by alkyl, alkoxy, halogen, CF₃, amino, substituted amino, cyano, or —S(O)ₙR_{SO}, in which R_{SO} is alkyl, aryl, or heteroaryl and n is 0, 1 or 2; or (3) an alkyl group as defined above that has both 1, 2, 3, 4 or 5 substituents as defined above and is also interrupted by 1-10 atoms as defined above. For example, the alkyl groups can be an alkyl hydroxy group, where any of the hydrogen atoms of the alkyl group are substituted with a hydroxyl group.

The term “alkyl group” as defined herein also includes cycloalkyl groups. The term “cycloalkyl group” as used herein is a non-aromatic carbon-based ring (i.e., carbocyclic) composed of at least three carbon atoms, and in some embodiments from three to 20 carbon atoms, having a single cyclic ring or multiple condensed rings. Examples of single ring cycloalkyl groups include, but are not limited to, cyclopropyl, cyclobutyl, cyclopentyl, cyclohexyl, cyclooctyl, and the like. Examples of multiple ring cycloalkyl groups include, but are not limited to, adamantanyl, bicyclo[2.2.1]heptane, 1,3,3-trimethylbicyclo[2.2.1]hept-2-yl, (2,3,3-trimethylbicyclo[2.2.1]hept-2-yl), or carbocyclic groups to which is fused an aryl group, for example indane, and the like. The term cycloalkyl group also includes a heterocycloalkyl group, where at least one of the carbon atoms of the ring is substituted with a heteroatom such as, but not limited to, nitrogen, oxygen, sulfur, or phosphorus.

The term “unsubstituted alkyl group” is defined herein as an alkyl group composed of just carbon and hydrogen.

The term “acyl” denotes a group —C(O)R_{CO}, in which R_{CO} is hydrogen, optionally substituted alkyl, optionally substituted cycloalkyl, optionally substituted heterocyclyl, optionally substituted aryl, and optionally substituted heteroaryl.

The term “aryl group” as used herein is any carbon-based aromatic group (i.e., aromatic carbocyclic) such as having a single ring (e.g., phenyl) or multiple rings (e.g., biphenyl), or multiple condensed (fused) rings (e.g., naphthyl or anthryl). These may include, but are not limited to, benzene, naphthalene, phenyl, etc.

The term “aryl group” also includes “heteroaryl group,” meaning a radical derived from an aromatic cyclic group (i.e., fully unsaturated) having 1, 2, 3, 4, 5, 6, 7, 8, 9, 10, 11, 12, 13, 14, or 15 carbon atoms and 1, 2, 3 or 4 heteroatoms selected from oxygen, nitrogen, sulfur, and phosphorus within at least one ring. In other words, heteroaryl groups are aromatic rings composed of at least three carbon atoms that has at least one heteroatom incorporated within the ring of the aromatic group. Such heteroaryl groups can have a single ring (e.g., pyridyl or furyl) or multiple condensed rings (e.g., indolizinyl, benzothiazolyl, or benzothienyl). Examples of heteroaryls include, but are not limited to, [1,2,4]oxadiazole, [1,3,4]oxadiazole, [1,2,4]thiadiazole, [1,3,4]thiadiazole, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, triazole, oxazole, thiazole, naphthyridine, and the like as well as N-oxide and N-alkoxy derivatives of nitrogen containing heteroaryl compounds, for example pyridine-N-oxide derivatives.

Unless otherwise constrained by the definition for the heteroaryl substituent, such heteroaryl groups can be optionally substituted with 1 to 5 substituents, typically 1 to 3 substituents selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1-3 substituents chosen from alkyl, carboxy, carboxyalkyl, aminocarbonyl, hydroxy, alkoxy, halogen, CF₃, amino, substituted amino, cyano, and —S(O)ₙR_{SO}, where R_{SO} is alkyl, aryl, or heteroaryl and n is 0, 1 or 2.

The aryl group can be substituted or unsubstituted. Unless otherwise constrained by the definition for the aryl substituent, such aryl groups can optionally be substituted with from 1 to 5 substituents, typically 1 to 3 substituents, selected from the group consisting of alkyl, alkenyl, alkynyl, alkoxy, aldehyde, cycloalkyl, cycloalkenyl, acyl, acylamino, acyloxy, amino, aminocarbonyl, alkoxycarbonylamino, azido, cyano, ester, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1-3 substituents chosen from alkyl, carboxy, carboxyalkyl, aminocarbonyl, hydroxy, alkoxy, halogen, CF₃, amino, substituted amino, cyano, and —S(O)ₙR₅₀, where R_{SO} is alkyl, aryl, or heteroaryl and n is 0, 1 or 2. In some embodiments, the term “aryl group” is limited to substituted or unsubstituted aryl and heteroaryl rings having from three to 30 carbon atoms.

The term “aralkyl group” as used herein is an aryl group having an alkyl group or an alkylene group as defined herein covalently attached to the aryl group. An example of an aralkyl group is a benzyl group. “Optionally substituted aralkyl” refers to an optionally substituted aryl group covalently linked to an optionally substituted alkyl group or alkylene group. Such aralkyl groups are exemplified by benzyl, phenylethyl, 3-(4-methoxyphenyl)propyl, and the like.

The term “heteroaralkyl” refers to a heteroaryl group covalently linked to an alkylene group, where heteroaryl and alkylene are defined herein. “Optionally substituted heteroaralkyl” refers to an optionally substituted heteroaryl group covalently linked to an optionally substituted alkylene group. Such heteroaralkyl groups are exemplified by 3-pyridylmethyl, quinolin-8-ylethyl, 4-methoxythiazol-2-ylpropyl, and the like.

The term “alkenyl group” refers to a monoradical of a branched or unbranched unsaturated hydrocarbon group typically having from 2 to 40 carbon atoms, more typically 2 to 10 carbon atoms and even more typically 2 to 6 carbon atoms and having 1-6, typically 1, double bond (vinyl). Typical alkenyl groups include ethenyl or vinyl (—CH═CH₂), 1-propylene or allyl (—CH₂CH═CH₂), isopropylene (—C(CH₃)═CH₂), bicyclo[2.2.1]heptene, and the like. When alkenyl is attached to nitrogen, the double bond cannot be alpha to the nitrogen.

The term “substituted alkenyl group” refers to an alkenyl group as defined above having 1, 2, 3, 4 or 5 substituents, and typically 1, 2, or 3 substituents, selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO— heteroaryl, —SO₂-alkyl, SO₂-aryl and —SO₂-heteroaryl. Unless otherwise constrained by the definition, all substituents may optionally be further substituted by 1, 2, or 3 substituents chosen from alkyl, carboxy, carboxyalkyl, aminocarbonyl, hydroxy, alkoxy, halogen, CF₃, amino, substituted amino, cyano, and —S(O)ₙR_{SO}, where R_{SO} is alkyl, aryl, or heteroaryl and n is 0, 1 or 2.

The term “cycloalkenyl group” refers to carbocyclic groups of from 3 to 20 carbon atoms having a single cyclic ring or multiple condensed rings with at least one double bond in the ring structure.

The term “alkynyl group” refers to a monoradical of an unsaturated hydrocarbon, typically having from 2 to 40 carbon atoms, more typically 2 to 10 carbon atoms and even more typically 2 to 6 carbon atoms and having at least 1 and typically from 1-6 sites of acetylene (triple bond) unsaturation. Typical alkynyl groups include ethynyl, (—C≡CH), propargyl (or prop-1-yn-3-yl, —CH₂C≡CH), and the like. When alkynyl is attached to nitrogen, the triple bond cannot be alpha to the nitrogen.

The term “substituted alkynyl group” refers to an alkynyl group as defined above having 1, 2, 3, 4 or 5 substituents, and typically 1, 2, or 3 substituents, selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1, 2, or 3 substituents chosen from alkyl, carboxy, carboxyalkyl, aminocarbonyl, hydroxy, alkoxy, halogen, CF₃, amino, substituted amino, cyano, and —S(O)ₙR_{SO}, where R_{SO} is alkyl, aryl, or heteroaryl and n is 0, 1 or 2.

The term “alkylene group” is defined as a diradical of a branched or unbranched saturated hydrocarbon chain, having 1, 2, 3, 4, 5, 6, 7, 8, 9, 10, 11, 12, 13, 14, 15, 16, 17, 18, 19 or 20 carbon atoms, typically 1-10 carbon atoms, more typically 1, 2, 3, 4, 5 or 6 carbon atoms. This term is exemplified by groups such as methylene (—CH₂—), ethylene (—CH₂CH₂—), the propylene isomers (e.g., —CH₂CH₂CH₂— and —CH(CH₃)CH₂—) and the like.

The term “substituted alkylene group” refers to: (1) an alkylene group as defined above having 1, 2, 3, 4, or 5 substituents selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO-heteroaryl, —SO₂-alkyl, —SO₂-aryl and —SO₂-heteroaryl. Unless otherwise constrained by the definition, all substituents may optionally be further substituted by 1, 2, or 3 substituents chosen from alkyl, carboxy, carboxyalkyl, aminocarbonyl, hydroxy, alkoxy, halogen, CF₃, amino, substituted amino, cyano, and —S(O)ₙR_{SO}, where R_{SO} is alkyl, aryl, or heteroaryl and n is 0, 1 or 2; or (2) an alkylene group as defined above that is interrupted by 1-20 atoms independently chosen from oxygen, sulfur and NRₐ—, where Rₐ is chosen from hydrogen, optionally substituted alkyl, cycloalkyl, cycloalkenyl, aryl, heteroaryl and heterocyclyl, or groups selected from carbonyl, carboxyester, carboxyamide and sulfonyl; or (3) an alkylene group as defined above that has both 1, 2, 3, 4 or 5 substituents as defined above and is also interrupted by 1-20 atoms as defined above. Examples of substituted alkylenes are chloromethylene (—CH(Cl)—), aminoethylene (—CH(NH₂)CH₂—), methylaminoethylene (—CH(NHMe)CH₂—), 2-carboxypropylene isomers (—CH₂CH(CO₂H)CH₂—, ethoxyethyl (—CH₂CH₂O—CH₂CH₂—), ethylmethylaminoethyl (—CH₂CH₂N(CH₃)CH₂CH₂—), and the like.

The term “alkoxy group” refers to the group R—O—, where R is an optionally substituted alkyl or optionally substituted cycloalkyl, or R is a group —Y—Z, in which Y is optionally substituted alkylene and Z is optionally substituted alkenyl, optionally substituted alkynyl; or optionally substituted cycloalkenyl, where alkyl, alkenyl, alkynyl, cycloalkyl and cycloalkenyl are as defined herein. Typical alkoxy groups are optionally substituted alkyl-O— and include, by way of example, methoxy, ethoxy, n-propoxy, iso-propoxy, n-butoxy, tert-butoxy, sec-butoxy, n-pentoxy, n-hexoxy, 1,2-dimethylbutoxy, trifluoromethoxy, and the like.

The term “alkylthio group” refers to the group R_{S}—S—, where R_{S} is as defined for alkoxy.

The term “aminocarbonyl” refers to the group —C(O)NR_{N}R_{N} where each R_{N} is independently hydrogen, alkyl, aryl, heteroaryl, heterocyclyl or where both R_{N} groups are joined to form a heterocyclic group (e.g., morpholino). Unless otherwise constrained by the definition, all substituents may optionally be further substituted by 1-3 substituents chosen from alkyl, carboxy, carboxyalkyl, aminocarbonyl, hydroxy, alkoxy, halogen, CF₃, amino, substituted amino, cyano, and —S(O)ₙR_{SO}, where R_{SO} is alkyl, aryl, or heteroaryl and n is 0, 1 or 2.

The term “acylamino” refers to the group —NR_{NCO}C(O)R where each R_{NCO} is independently hydrogen, alkyl, aryl, heteroaryl, or heterocyclyl. Unless otherwise constrained by the definition, all substituents may optionally be further substituted by 1-3 substituents chosen from alkyl, carboxy, carboxyalkyl, aminocarbonyl, hydroxy, alkoxy, halogen, CF₃, amino, substituted amino, cyano, and —S(O)ₙR_{SO}, where R_{SO} is alkyl, aryl, or heteroaryl and n is 0, 1 or 2.

The term “acyloxy” refers to the groups —O(O)C-alkyl, —O(O)C-cycloalkyl, —O(O)C-aryl, —O(O)C-heteroaryl, and —O(O)C-heterocyclyl. Unless otherwise constrained by the definition, all substituents may be optionally further substituted by alkyl, carboxy, carboxyalkyl, aminocarbonyl, hydroxy, alkoxy, halogen, CF₃, amino, substituted amino, cyano, and —S(O)ₙR_{SO}, where R_{SO} is alkyl, aryl, or heteroaryl and n is 0, 1 or 2.

The term “aryloxy group” refers to the group aryl-O— wherein the aryl group is as defined above, and includes optionally substituted aryl groups as also defined above.

The term “heteroaryloxy” refers to the group heteroaryl-O—.

The term “amino” refers to the group —NH₂.

The term “substituted amino” refers to the group —NR_{w}R_{w} where each R_{w} is independently selected from the group consisting of hydrogen, alkyl, cycloalkyl, carboxyalkyl (for example, benzyloxycarbonyl), aryl, heteroaryl and heterocyclyl provided that both R_{w} groups are not hydrogen, or a group —Y—Z, in which Y is optionally substituted alkylene and Z is alkenyl, cycloalkenyl, or alkynyl. Unless otherwise constrained by the definition, all substituents may optionally be further substituted by 1-3 substituents chosen from alkyl, carboxy, carboxyalkyl, aminocarbonyl, hydroxy, alkoxy, halogen, CF₃, amino, substituted amino, cyano, and —S(O)ₙR_{SO}, where R_{SO} is alkyl, aryl, or heteroaryl and n is 0, 1 or 2.

The term “carboxy” refers to a group —C(O)OH. The term “carboxyalkyl group” refers to the groups —C(O)O-alkyl or —C(O)O-cycloalkyl, where alkyl and cycloalkyl, are as defined herein, and may be optionally further substituted by alkyl, alkenyl, alkynyl, alkoxy, halogen, CF₃, amino, substituted amino, cyano, and —S(O)ₙR_{SO}, in which R_{SO} is alkyl, aryl, or heteroaryl and n is 0, 1 or 2.

The terms “substituted cycloalkyl group” or “substituted cycloalkenyl group” refer to cycloalkyl or cycloalkenyl groups having 1, 2, 3, 4 or 5 substituents, and typically 1, 2, or 3 substituents, selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO— heteroaryl, —SO₂-alkyl, SO₂-aryl and —SO₂-heteroaryl. Unless otherwise constrained by the definition, all substituents may optionally be further substituted by 1, 2, or 3 substituents chosen from alkyl, carboxy, carboxyalkyl, aminocarbonyl, hydroxy, alkoxy, halogen, CF₃, amino, substituted amino, cyano, and —S(O)ₙR_{SO}, where R_{SO} is alkyl, aryl, or heteroaryl and n is 0, 1 or 2.

The term “conjugated group” is defined as a linear, branched or cyclic group, or combination thereof, in which p-orbitals of the atoms within the group are connected via delocalization of electrons and wherein the structure can be described as containing alternating single and double or triple bonds and may further contain lone pairs, radicals, or carbenium ions. Conjugated cyclic groups may comprise both aromatic and non-aromatic groups, and may comprise polycyclic or heterocyclic groups, such as diketopyrrolopyrrole. Ideally, conjugated groups are bound in such a way as to continue the conjugation between the thiophene moieties they connect. In some embodiments, “conjugated groups” is limited to conjugated groups having three to 30 carbon atoms.

The term “halogen,” “halo,” or “halide” may be referred to interchangeably and refer to fluoro, bromo, chloro, and iodo.

The term “heterocyclyl” refers to a monoradical saturated or partially unsaturated group having a single ring or multiple condensed rings, having from 1 to 40 carbon atoms and from 1 to 10 hetero atoms, typically 1, 2, 3 or 4 heteroatoms, selected from nitrogen, sulfur, phosphorus, and/or oxygen within the ring. Heterocyclic groups can have a single ring or multiple condensed rings, and include tetrahydrofuranyl, morpholino, piperidinyl, piperazino, dihydropyridino, and the like.

Unless otherwise constrained by the definition for the heterocyclyl substituent, such heterocyclyl groups can be optionally substituted with 1, 2, 3, 4 or 5, and typically 1, 2 or 3 substituents, selected from the group consisting of alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, heteroarylthio, heterocyclylthio, thiol, alkylthio, aryl, aryloxy, heteroaryl, aminosulfonyl, aminocarbonylamino, heteroaryloxy, heterocyclyl, heterocyclooxy, hydroxyamino, alkoxyamino, nitro, —SO-alkyl, —SO-aryl, —SO— heteroaryl, —SO₂-alkyl, —SO₂-aryl and —SO₂-heteroaryl. Unless otherwise constrained by the definition, all substituents may optionally be further substituted by 1-3 substituents chosen from alkyl, carboxy, carboxyalkyl, aminocarbonyl, hydroxy, alkoxy, halogen, CF₃, amino, substituted amino, cyano, and —S(O)ₙR_{SO}, where R_{SO} is alkyl, aryl, or heteroaryl and n is 0, 1 or 2.

The term “thiol” refers to the group —SH. The term “substituted alkylthio” refers to the group —S— substituted alkyl. The term “arylthiol group” refers to the group aryl-S—, where aryl is as defined as above. The term “heteroarylthiol” refers to the group —S— heteroaryl wherein the heteroaryl group is as defined above including optionally substituted heteroaryl groups as also defined above.

The term “sulfoxide” refers to a group —S(O)R_{SO}, in which R_{SO} is alkyl, aryl, or heteroaryl. The term “substituted sulfoxide” refers to a group —S(O)R_{SO}, in which R_{SO} is substituted alkyl, substituted aryl, or substituted heteroaryl, as defined herein. The term “sulfone” refers to a group —S(O)₂R_{SO}, in which R_{SO} is alkyl, aryl, or heteroaryl. The term “substituted sulfone” refers to a group —S(O)₂R_{SO}, in which R_{SO} is substituted alkyl, substituted aryl, or substituted heteroaryl, as defined herein.

The term “keto” refers to a group —C(O)—. The term “thiocarbonyl” refers to a group —C(S)—.

As used herein, the term “room temperature” is 20° C. to 25° C.

Disclosed are compounds, compositions, and components that can be used for, can be used in conjunction with, can be used in preparation of, or are products of the disclosed methods and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this disclosure including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

A weight percent of a component, unless specifically stated to the contrary, is based on the total weight of the formulation or composition in which the component is included.

Organic semiconductors as functional materials may be used in a variety of applications including, for example, printed electronics, organic transistors, including organic thin-film transistors (OTFTs) and organic field-effect transistors (OFETs), organic light-emitting diodes (OLEDs), organic integrated circuits, organic solar cells, and disposable sensors. Organic transistors may be used in many applications, including smart cards, security tags, and the backplanes of flat panel displays. Organic semiconductors may substantially reduce cost compared to inorganic counterparts, such as silicon. Depositing OSCs from solution may enable fast, large-area fabrication routes such as various printing methods and roll-to-roll processes.

Organic thin-film transistors are particularly interesting because their fabrication processes are less complex as compared with conventional silicon-based technologies. For example, OTFTs generally rely on low temperature deposition and solution processing, which, when used with semiconducting conjugated materials, can achieve valuable technological attributes, such as compatibility with simple-write printing techniques, general low-cost manufacturing approaches, and flexible plastic substrates. Other potential applications for OTFTs include flexible electronic papers, sensors, memory devices (e.g., radio frequency identification cards (RFIDs)), remote controllable smart tags for supply chain management, large-area flexible displays, and smart cards.

As provided herein, successful synthesis of FT4-based small molecules are demonstrated and may be useful as precursor monomers for further novel OSC polymer synthesis processing, or themselves be directly utilized as organic semiconducting materials for various electronic and photonic applications (e.g., OTFT, OLED, OPV devices) or as fluorescent materials in other applications.

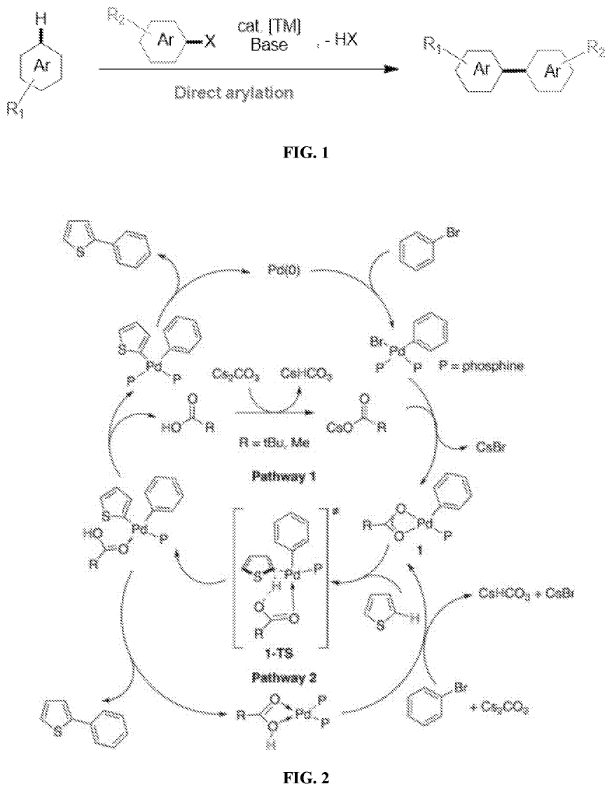

Turning now to FIG. 1, which illustrates a general Pd-catalyzed aryl cross-coupling reaction. Coupling of aryl halides with catalytically activated aryl C—H bonds provides an environmentally benign and atom-economical alternative to standard cross-coupling reactions. Direct (hetero)arylation, involves coupling of a pre-functionalized arene-bearing leaving group with an arene C—H bond. Regioselectivity in these reactions is dependent on the arene systems used. A base may also be added to assist in C—H bond activation and neutralize the stoichiometric amount of acid formed. In the direct arylation of FIG. 1, ‘Ar’ is an aryl group, R₁ and R₂ are, for example, an alkyl group, an alkenyl group, an alkynyl group, an alkylene group, or combination thereof (substituted or unsubstituted), and ‘X’ may be a halide or the like having similar functionality.

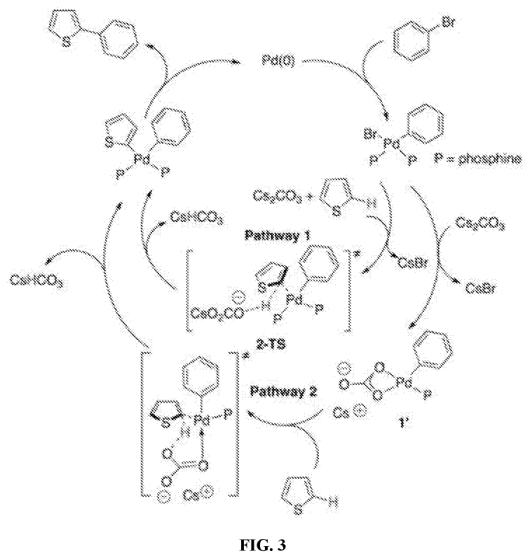

FIG. 2 represents a general catalytic cycle for direct (hetero)arylation between thiophene and bromobenzene with a carboxylate additive, while FIG. 3 illustrates a catalytic cycle for direct (hetero)arylation between thiophene and bromobenzene without a carboxylate additive.

The mechanism by which C—H activation includes electrophilic aromatic substitution, Heck-type coupling and concerted metalation-deprotonation (CMD). Most heterocycles, such as thiophenes and indoles, are believed to follow a base-assisted CMD pathway. During the process, carboxylate or carbonate anions coordinate to the metal center (in most cases Pd) in-situ and assist in the deprotonation transition state. Among numerous arenes and heteroarenes, thiophene substrates have demonstrated high reactivity toward C—H bond activation when appropriately substituted with electron-rich or electron-deficient groups. This is further explained below.

Two catalytic cycles for a CMD coupling of bromobenzene and thiophene using a palladium/phosphine catalytic system and cesium carbonate are shown in FIGS. 2 and 3. Under carboxylate-mediated conditions of FIG. 2, oxidative addition of the carbon-halogen bond is followed by exchange of the halogen ligand for the carboxylate anion to form complex 1. With assistance from the carboxylate ligand, complex 1 then deprotonates the thiophene substrate while simultaneously forming a metal-carbon bond and goes through transition state 1-TS. The phosphine ligands, or the solvent, can re-coordinate to the metal center following Pathway 1, or the carboxylate group can remain coordinated throughout the entire process, as in Pathway 2. Finally, reductive elimination renders the aryl coupled product.

In absence of a carboxylate additive, after oxidative addition of the aryl bromide, the reaction follows one of the two general pathways shown in FIG. 3. If a bidentate phosphine is employed, C—H activation of thiophene follows Pathway 1, where deprotonation is assisted intermolecularly (2-TS). When a monodentate phosphine is used, the reaction follows either Pathway 1 or Pathway 2. The latter mechanism (Pathway 2) most closely resembles Pathway 2 in FIG. 2, where the carbonate coordinates to the metal center to give the Zwitterionic species 1′. From here, deprotonation occurs intramolecularly through transition state 1′-TS. Reductive elimination then renders 2-phenylthiophene.

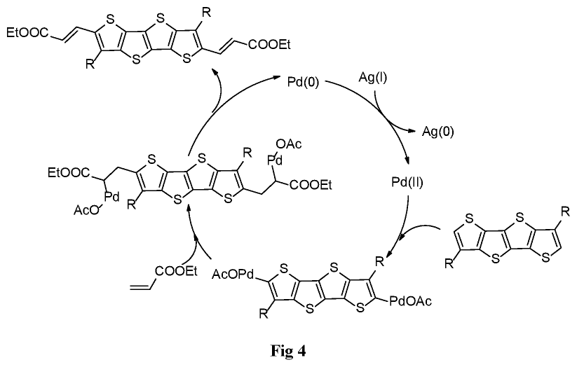

FIG. 4 illustrates a mechanism of direct alkenylation with a silver (Ag) oxidant. Initially, alkyl substituted tetrathienoacene reacts with palladium catalyst (Pd (II)) through an electrophilic C—H activation pathway at the C2 position of tetrathienoacene to afford a palladation intermediate. After a subsequent addition of alkenyl C═C double bond to form a Pd—C bond, and thereafter a β-H elimination, a final C2-alkenylated product is formed, which undergoes a classical Heck-type reaction. Finally, the Pd(0) is then re-oxidized to Pd(II) by the Ag₂CO₃.

In organic electronics, thiophene-based organic semiconducting materials may be used in organic thin film transistors (TFTs), organic photovoltaics (OPVs), and organic light-emitting diodes (OLEDs). Traditionally synthesized using Stille coupling, the disclosure herein provides for an alternative mechanism for developing environmentally benign and low-cost organic semiconducting materials. Specifically, novel tetrathienoacene (FT4)-based small molecules (having excellent coplanarity, strong π-π intermolecular stacking, and high charge mobility) were synthesized by Pd-catalyzed direct (hetero)arylation for materials in OTFT and OPV devices.

In some embodiments, the tetrathienoacene (FT4)-based small molecules described herein have a molecular weight in a range of 500 Da to 20000 Da, or 750 Da to 15000 Da, or 1000 Da to 12500 Da, or 1250 Da to 10000 Da, or 1500 Da to 7500 Da, or any value or range disclosed therebetween. In some embodiments, the tetrathienoacene (FT4)-based small molecules described herein have a molecular weight of 500 Da, or 600 Da, 700 Da, or 800 Da, or 900 Da, or 1000 Da, or 1100 Da, or 1200 Da, or 1300 Da, or 1400 Da, or 1500 Da, or 1600 Da, or 1700 Da, or 1800 Da, or 1900 Da, or 2000 Da, or 2100 Da, or 2200 Da, or 2300 Da, or 2400 Da, or 2500 Da, or 2600 Da, or 2700 Da, or 2800 Da, or 2900 Da, or 3000 Da, or 3500 Da, or 4000 Da, or 4500 Da, or 5000 Da, or 5500 Da, or 6000 Da, or 6500 Da, or 7000 Da, or 7500 Da, or 8000 Da, or 8500 Da, or 9000 Da, or 9500 Da, or 10000 Da, or 10500 Da, or 11000 Da, or 11500 Da, or 12000 Da, or 12500 Da, or 13000 Da, or 14000 Da, or 15000 Da, or 16000 Da, or 17000 Da, or 18000 Da, or 19000 Da, or 20000 Da, or any value of range disclosed therein.

### Examples

The embodiments described herein will be further clarified by the following examples.

In order to improve cost efficiency and minimize environmental impact, a highly efficient catalysis system is identified for providing high regioselectivity and turnover frequency and turnover numbers. Several components were tested, including: catalyst, additive (e.g., to promote deprotonation of aromatic hydrogens), base (e.g., to assist in removing bromide and promote oxidative addition of aromatic bromides), ligand (e.g., to stabilize Pd species in order to prevent transformation into Pd black, which does not participate in the catalytic cycle), oxidant, solvent, reaction time, and temperature.

### Example 1—Direct Arylation Between FT4 and Methyl Bromothiophene

Effects of catalyst, additive, base, ligand, solvent, reaction time, and temperature on yield in the direct arylation between 3-alkyl-FT4 and 2-bromo-5-methyl-thiophene (Reaction 1) are shown in Table 1. In order to reduce optimization turnaround time, a mono-Br substituted small molecule 2-bromo-5-methyl-thiophene was selected to react with FT4 monomer, since small molecules are easier to separate and characterize than high molecular weight polymers.

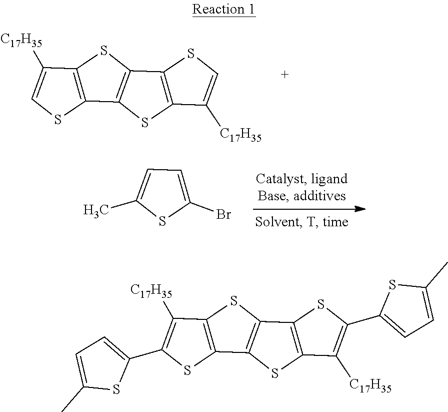

TABLE 1

| | | Additive | | | | Temp | Yield |
|---|---|---|---|---|---|---|---|
| Entry No. | Catalyst (mol. %) | (mol. %) | Base (eq) | Solvent | Time (hrs) | (° C.) | (%) |
| 1 | Pd(OAc)₂ (4) | PivOH (30) | K₂CO₃ (2.5) | DMAc | 24 | 110 | 17 |
| 2 | PdC1₂ (4) | PivOH (30) | K₂CO₃ (2.5) | DMAc | 24 | 110 | 11 |
| 3 | Pd(O₂CCF₃)₂ (4) | PivOH (30) | K₂CO₃ (2.5) | DMAc | 24 | 110 | 10 |
| 4 | C₈H₁₂B₂F₈N₄Pd (4) | PivOH (30) | K₂CO₃ (2.5) | DMAc | 24 | 110 | 14 |
| 5 | Pd(PPh₃)₄ (4) | PivOH (30) | K₂CO₃ (2.5) | DMAc | 24 | 110 | 17 |
| 6 | Pd/C (4) | PivOH (30) | K₂CO₃ (2.5) | DMAc | 24 | 110 | NR |
| 7 | Pd₂(dba)₃ (4); | PivOH (30) | K₂CO₃ (2.5) | DMAc | 24 | 110 | 13 |
| | PPh₃ (4) | | | | | | |
| 8 | Pd(OAc)₂ (4) | PivOH (30) | K₂CO₃ (2.5) | Toluene | 24 | 110 | 28 |
| 9 | Pd(OAc)₂ (4) | PivOH (30) | K₂CO₃ (2.5) | THF | 24 | 60 | NR |
| 10 | Pd(OAc)₂ (4) | PivOH (30) | K₂CO₃ (2.5) | DMF | 24 | 110 | 22 |
| 11 | Pd₂(dba)₃ (10); | PivOH (30) | Cs₂CO₃ (2.5) | Toluene | 24 | 100 | 24 |
| | P-(o-MeOPh)₃ (20) | | | | | | |
| 12 | Pd₂(dba)₃ (15); | PivOH (30) | Cs₂CO₃ (2) | Toluene | 24 | 100 | 17 |
| | P-(o-MeOPh)₃ (30) | | | | | | |
| 13 | Pd₂(dba)₃ (15); | PivOH (30) | Cs₂CO₃ (2) | Toluene | 18 | 100 | 20 |
| | P-(o-MeOPh)₃ (30) | | | | | | |
| 14 | Pd₂(dba)₃ (15); | PivOH (30) | Cs₂CO₃ (2) | Toluene | 12 | 100 | 31 |
| | P-(o-MeOPh)₃ (30) | | | | | | |
| 15 | Pd₂(dba)₃ (15); | PivOH (30) | Cs₂CO₃ (2) | Toluene | 6 | 100 | 29 |
| | P-(o-MeOPh)₃ (30) | | | | | | |

Excluding Entry 6, Entries 1-5 and 7-15 all included ligands. Ligands were part of the catalyst. For example, in Entry 3, O₂CCF₃ was used as the ligand; in Entries 8-10, OAc was used; and in Entries 11-15, dba was used as the ligand, with extra ligands (e.g., P-(o-MeOPh)₃) specifically added as a replacement for existing ligands to offer better results. Such use of ligands are analogously shown throughout Table 1 and other examples disclosed herein.

Entry 1 represents a baseline direct arylation catalysis system comprising Pd(OAc)₂ as the catalyst, no ligand, pivalic acid (PivOH) as the additive, K₂CO₃ as the base, dimethylacetamide (DMAc) as solvent. “Ac” refers to acetamide and “dba” refers to dibenzylideneacetone. Reaction 1 is carried out at 110° C. for 24 hrs. and yielded 17% of the target product. Thereafter, Entries 2-7 were modified in the type of catalyst utilized (with the concentration remaining the same as in Entry 1), while keeping all other reaction conditions the same as the baseline case. Thereafter, in Entries 8-10, the type of solvent used was varied, with all other reaction conditions remaining the same as the baseline case (apart from Entry 9). Variation of solvent had a more significant impact on yield, as replacing DMAc with toluene improved yield from 17% (Entry 1) to 28% (Entry 8). Dimethylformamide (DMF) also showed improvement over DMAc, resulting in a yield of 22% (Entry 10). Finally, the influence of ligands and reaction time were investigated in Entries 11-15, with Entry 14 exhibiting the highest yield in toluene. In addition to the examples described herein, some embodiments may include the following solvents, catalysts, additives, oxidants, and ligands, as shown in Table 2.

TABLE 2

| | |
|---|---|
| Catalysts | PdCl₂(MeCN)₂ |
| | Pd₂(dba)₃ · CHCl₃ |
| | Herrmann-Beller catalyst |
| Ligands | PCy₃-HBF₄ |
| | tricyclohexylphosphonium tetrafluoroborate |
| | bis(tert-butyl) methylphosphonium tetrafluoroborate |
| Oxidants | Cu(OAc)₂ |
| | Ag₂CO₃ |
| | FeCl₃ |
| Additives | Neodecanoic acid (NDA) |
| | 2,2-diethylhexanoic acid (DEHA) |
| | CH₃COOK |
| Solvents | p-xylene |
| | mesitylene |
| | chlorobenzene |
| | o-dichlorobenzene |
| | 1,2,4-trichlorobenzene |
| | 1-chloronaphthalene |

Without being bound by theory, fused thiophene 3-alkyl-FT4 appears to have low reactivity towards Pd-catalyzed direct C—H coupling. Even after long reaction times, large quantities of unreacted starting materials were found in reaction mixtures.

### Example 2—Direct Alkenylation Between FT4 and Alkenes

Effects of catalyst, oxidant (e.g., to oxidize Pd(0) to Pd(II), see FIG. 4), solvent, reaction time, and temperature on yield in the direct arylation between 3-alkyl-FT4 and ethyl acrylate (Reaction 2) are shown in Table 3.

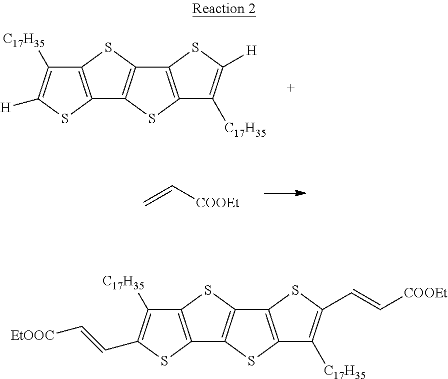

TABLE 3

| | | | | | Temp | Yield |
|---|---|---|---|---|---|---|
| Entry No. | Catalyst (mol. %) | Oxidant (eq) | Solvent | Time (hrs) | (° C.) | (%) |
| 16 | Pd(OAc)₂ (20) | AgOAc (3) | Benzotrifluoride | 12 | 100 | 60% |
| 17 | Pd(OAc)₂ (20) | AgOAc (3) | HFIP | 12 | 100 | NR |
| 18 | Pd(OAc)₂ (20) | AgOAc (3) | DCE | 12 | 100 | 40% |
| 19 | Pd(OAc)₂ (20) | AgOAc (3) | DME | 12 | 100 | 58% |
| 20 | Pd(OAc)₂ (20) | AgOAc (3) | Toluene | 12 | 100 | 67% |
| 21 | Pd(OAc)₂ (20) | AgOAc (3) | Hexafluorobenzene | 12 | 100 | 66% |
| 22 | Pd(OAc)₂ (20) | AgOAc (3) | 1,4-dioxane | 12 | 100 | 62% |
| 23 | Pd(OAc)₂ (20) | AgOAc (3) | Mesitylene | 12 | 100 | 64% |
| 24 | Pd(OAc)₂ (20) | AgOAc (3) | Chlorobenzene | 12 | 100 | 43% |
| 25 | PdCl₂ (20) | AgOAc (3) | Toluene | 12 | 100 | 6% |
| 26 | PdCO₂(CF₃)₂(20) | AgOAc (3) | Toluene | 12 | 100 | 62% |
| 27 | Tetrakis (acetonitrile) | AgOAc (3) | Toluene | 12 | 100 | 38% |
| | palladium (II) | | | | | |
| | tetrafluoroborate (20) | | | | | |
| 28 | Pd₂(dba)₃ (20) | AgOAc (3) | Toluene | 12 | 100 | 32% |
| 29 | Pd(OAc)₂ (20) | AgOAc (3) | Toluene | 12 | 60 | 64% |
| 30 | Pd(OAc)₂ (20) | AgOAc (3) | Toluene | 12 | 80 | 68% |
| 31 | Pd(OAc)₂ (20) | AgOAc (3) | Toluene | 12 | 120 | 63% |
| 32 | Pd(OAc)₂ (15) | AgOAc (3) | Toluene | 12 | 80 | 51% |
| 33 | Pd(OAc)₂ (10) | AgOAc (3) | Toluene | 12 | 80 | 48% |
| 34 | Pd(OAc)₂ (5) | AgOAc (3) | Toluene | 12 | 80 | 23% |
| 35 | Pd(OAc)₂ (20) | — | Toluene | 12 | 80 | 19% |
| 36 | Pd(OAc)₂ (20) | AgOAc (1) | Toluene | 12 | 80 | 56% |
| 37 | Pd(OAc)₂ (20) | AgOAc (2) | Toluene | 12 | 80 | 55% |
| 38 | Pd(OAc)₂ (20) | AgOAc (4) | Toluene | 12 | 80 | 70% |
| 39 | Pd(OAc)₂ (20) | Ag₂O (3) | Toluene | 12 | 80 | 15% |
| 40 | Pd(OAc)₂ (20) | CuCl₂ (3) | Toluene | 12 | 80 | 10% |
| 41 | Pd(OAc)₂ (20) | Cu(OAc)₂ (3) | Toluene | 12 | 80 | 15% |
| 42 | Pd(OAc)₂ (20) | V₂O₅ (3) | Toluene | 12 | 80 | 11% |
| 43 | Pd(OAc)₂ (20) | PhI(OAc)₂ (3) | Toluene | 12 | 80 | trace |
| 44 | Pd(OAc)₂ (20) | Ag₂CO₃ (3) | Toluene | 12 | 80 | 72% |

In order to improve conversion rate (i.e., yield), electron-deficient alkenes were selected as the coupling partner due to their good reactivity towards Pd-catalyzed C—C cross coupling. As shown in Table 3, 3-alkyl-FT4 demonstrated much better reactivity towards alkenes, as compared with methyl bromothiophenes (Table 1). Without being bound by theory, one reason might be due to the C—Pd species being more prone to migration insertion with electron-deficient olefins. From Table 3, Entry 44 provided the highest yield at 72%; thus, using these reaction conditions, other electron-deficient alkenes (e.g., methyl acylate, ethyl acrylate, styrene, etc.) were reacted using direct arylation with 3-alkyl-FT4 (analogous to Reaction 2) to synthesize the compounds of Table 4.

TABLE 4

| Compound | Structure | Yield (%) |
|---|---|---|
| 3a | 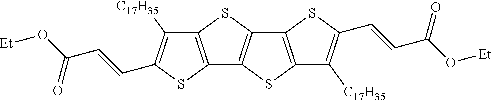 | 70 |
| 3b | 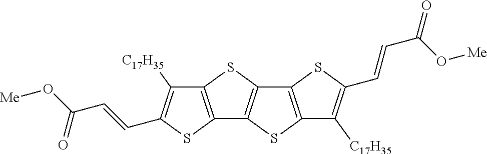 | 68 |
| 3c | 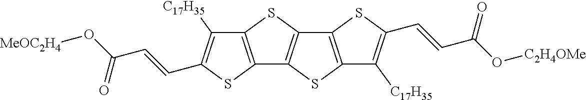 | 76 |
| 3d | 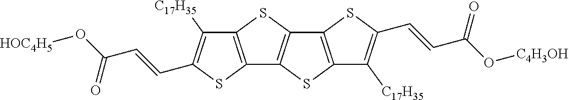 | 76 |
| 3e | 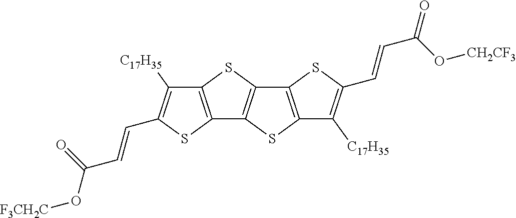 | 75 |
| 3f | 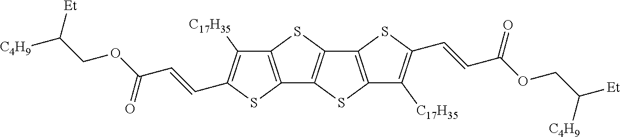 | 66 |
| 3g | 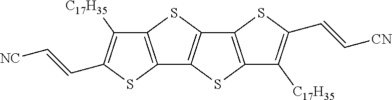 | 58 |
| 3h | 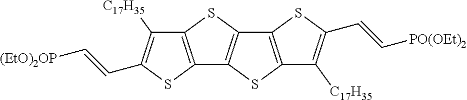 | 56 |
| 3i | 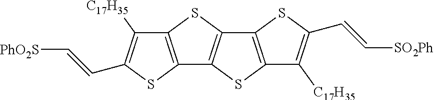 | 60 |
| 3j | 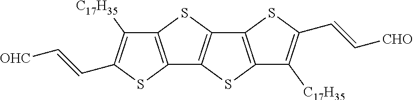 | 45 |
| 3k | 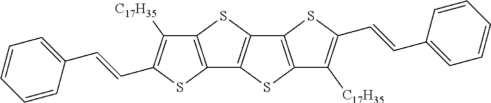 | 81 |
| 3l | 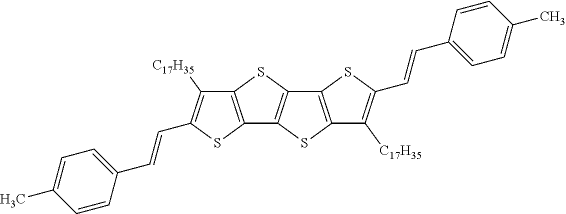 | 82 |
| 3m | 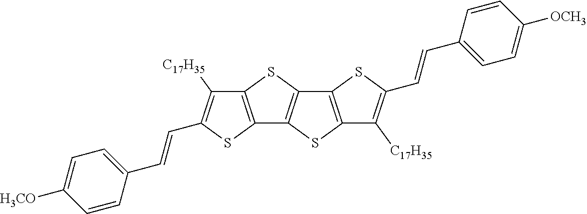 | 81 |
| 3n | 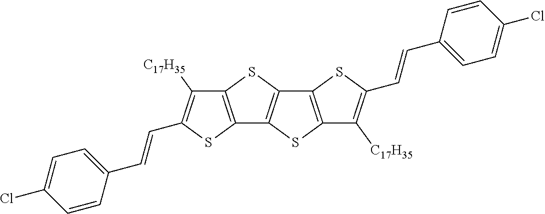 | 73 |
| 3o | 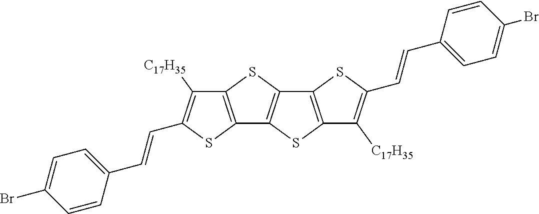 | 71 |
| 3p | 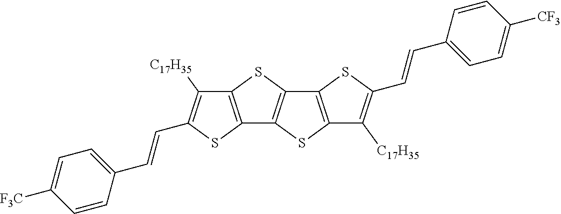 | 81 |
| 3q | 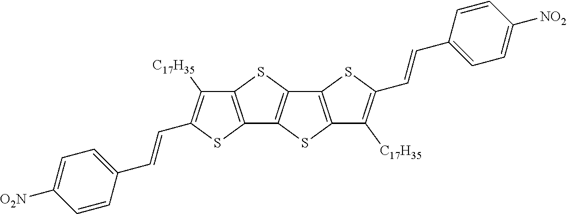 | 77 |
| 3r | 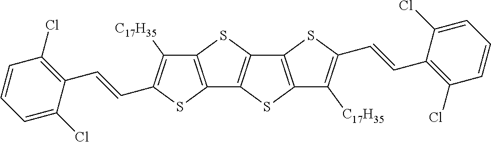 | 77 |
| 3s | 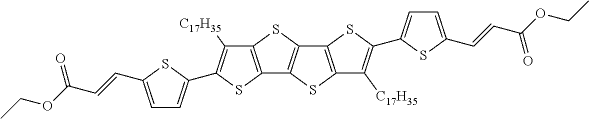 | — |
| 3t | 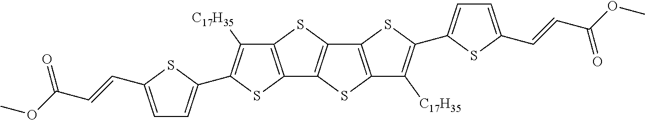 | — |
| 3u | 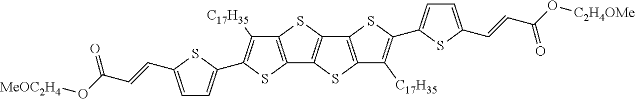 | — |
| 3v | 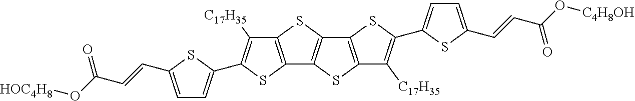 | — |
| 3w | 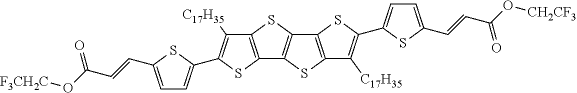 | — |
| 3x | 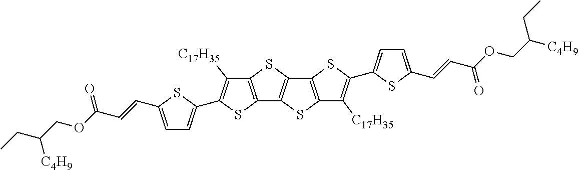 | — |
| 3y | 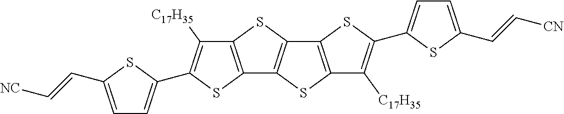 | — |
| 3z | 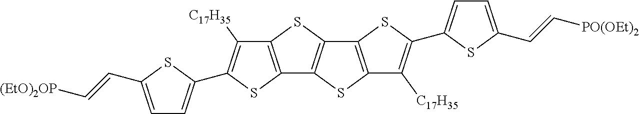 | — |
| 3aa | 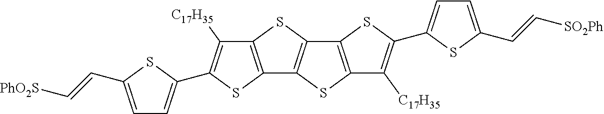 | — |
| 3bb | 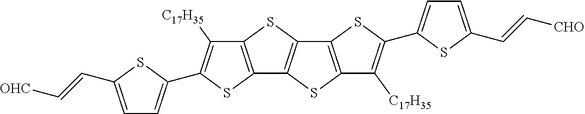 | — |
| 3cc | 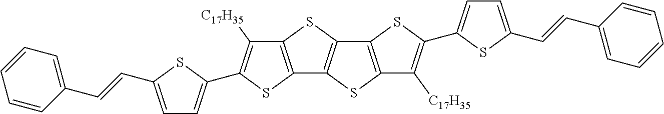 | — |
| 3dd | 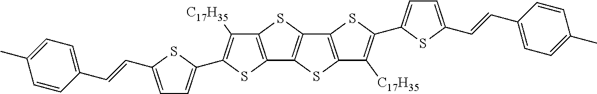 | — |
| 3ee | 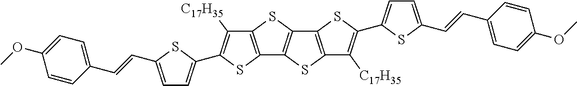 | — |
| 3ff | 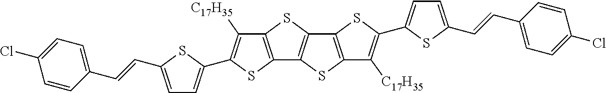 | — |
| 3gg | 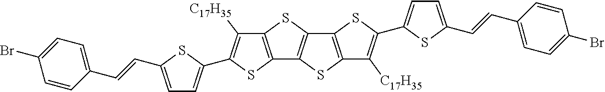 | — |
| 3hh | 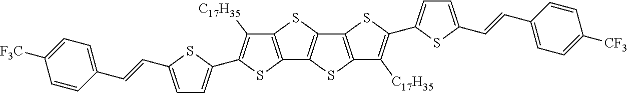 | — |
| 3ii | 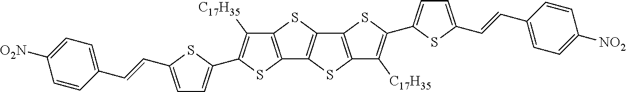 | — |
| 3jj | 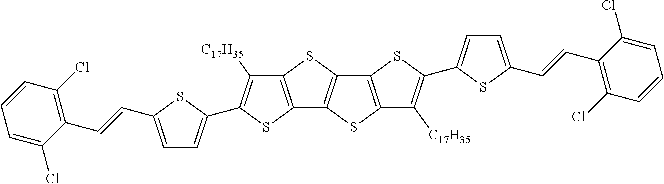 | — |

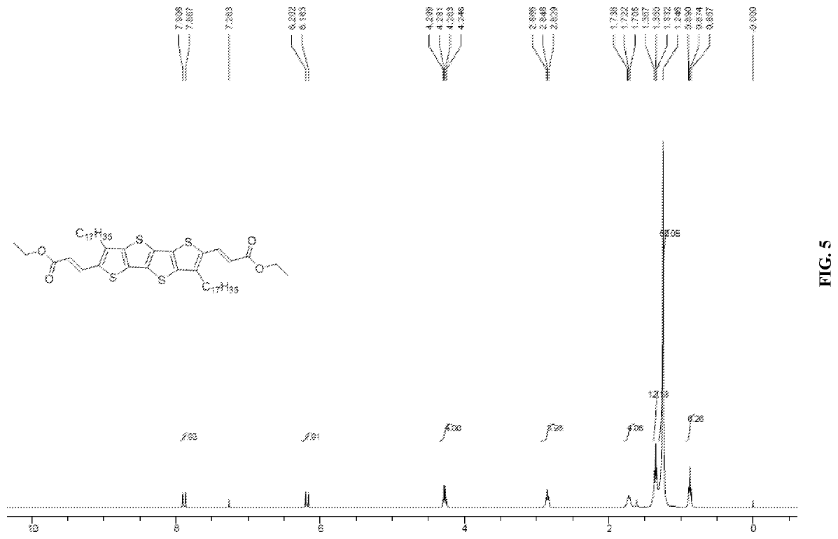

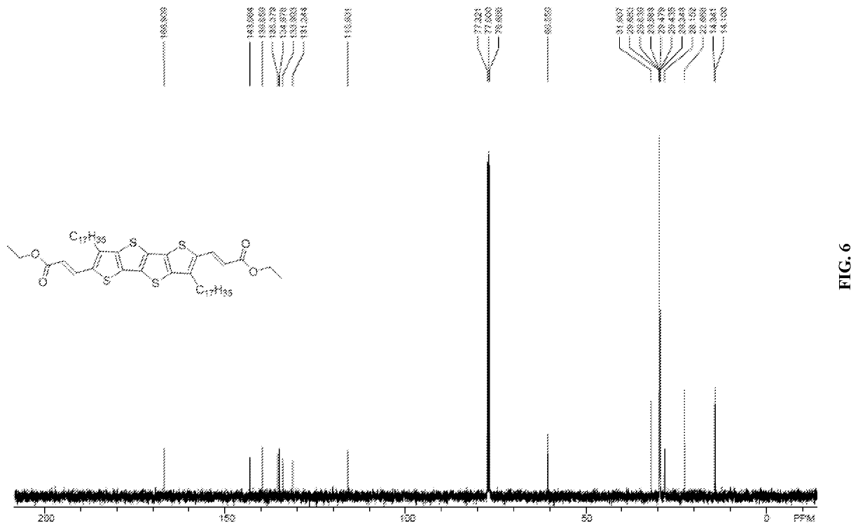

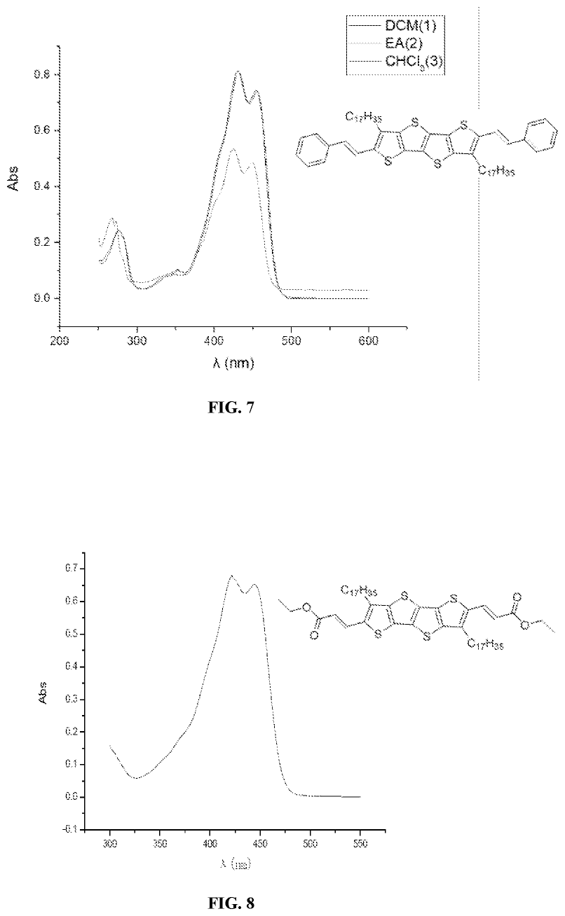

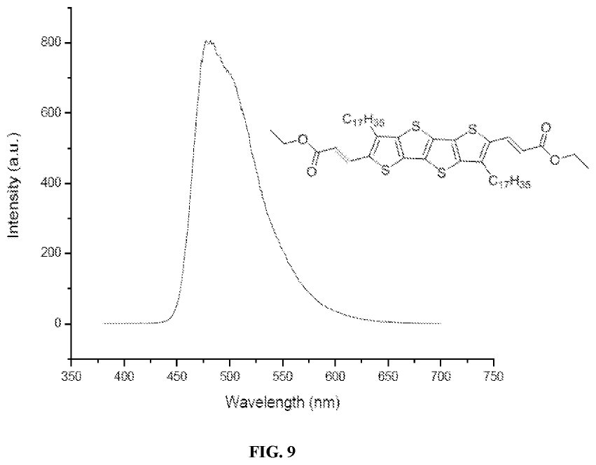

The product of Reaction 2 (Compound 3a) was characterized using proton nuclear magnetic resonance (¹H-NMR; Bruker 400 MHz spectrometer in CDCl₃) (FIG. 5), carbon-13 nuclear magnetic resonance (¹³C-NMR; Bruker 100 MHz spectrometer in CDCl₃) (FIG. 6), ultraviolet-visible absorption (UV-Vis; Shunyu Hengping UV 2400 spectrometer) (FIG. 8), and fluorescence spectroscopy (Agilent Gary Eclipse fluorescence spectrometer) (FIG. 9). Moreover, Compound 3k was also characterized using UV-Vis absorption (FIG. 7).

For Compound 3a, characteristic peaks in the ¹H-NMR spectra (FIG. 5) appear at (1) δ 7.89 (d, J=15.6 Hz, 2H, C═C—H); (2) δ 6.18 (d, J=15.6 Hz, 2H, C═C—H); (3) δ 4.27 (q, J=7.2 Hz, 4H, —OCH₂); (4) δ 2.85 (t, J=7.2 Hz, 4H); (5) δ 1.75-1.69 (m, 4H); (6) δ 1.39-1.31 (m, 12H); (7) δ 1.27-1.21 (m, 50H); and (8) 0.87 (t, J=6.4 Hz, 6H). For Compound 3a, characteristic peaks in the ¹³C NMR spectra (FIG. 6) appear at δ 166.91, 143.06, 139.66, 135.38, 134.98, 133.99, 131.24, 115.93, 60.55, 31.91, 29.68, 29.64, 29.59, 29.48, 29.44, 29.35, 28.15, 22.67, 14.34, and 14.10. For Compound 3a, UV-vis absorption (FIG. 8) at λ_{abs}=423 nm and 445 nm result in an ε^{b} of 5.55 and 5.58, respectively. For Compound 3a, a characteristic peak is observed at λₑₘ=482 nm in the fluorescence spectra of FIG. 9.

For Compound 3k, UV-vis absorption (FIG. 7) at λ_{abs}=430 nm and 454 nm result in an ε^{b} of 8.14 and 7.44, respectively, in DCM. For a λ_{abs}=425 nm and 449 nm, an of 5.33 and 4.85, respectively, is observed in ethyl alcohol (EA). For a λ_{abs}=431 nm and 456 nm, an of 8.10 and 7.37, respectively, is observed in CHCl₃.

### Example 3—Direct Arylation Polymerization Between FT4 and Dibromo-DPP

Effects of catalyst, additive, base, solvent, reaction time, temperature, and ligand on molecular weight in the direct arylation polymerization of FT4 and dibromo-diketopyrrolopyrrole (DPP) (Reaction 3) are shown in Tables 4 and 5.

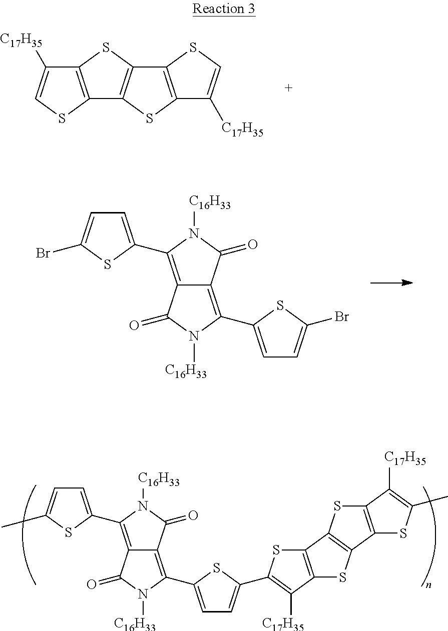

TABLE 5

| Entry | Catalyst | Additive | | | Time | Temp | Ligand |
|---|---|---|---|---|---|---|---|
| No. | (mol. %) | (mol. %) | Base (eq) | Solvent | (hrs) | (° C.) | (mol. %) |
| 45 | Pd₂(dba)₃ (2) | PivOH (30) | Cs₂CO₃ (2) | Toluene | 12 | 100 | P-(o-MeOPh)₃ (3) |
| 46 | Pd₂(dba)₃ (1.5) | PivOH (30) | Cs₂CO₃ (2) | DMAc | 12 | 100 | P-(o-MeOPh)₃ (3) |
| 47 | Pd(OAc)₂ (2) | PivOH (30) | Cs₂CO₃ (2.5) | DMAc | 48 | 100 | — |

TABLE 6

| | Number | | |
|---|---|---|---|
| | Avg | | Polydispersity |
| | Molecular | Molecular | Index |
| Entry | Wt. (Mₙ) | Wt. (M_{w}) | (M_{w}/Mₙ) |
| No. | (Da) | (Da) | (PDI) |
| 45 | 1690 | 2791 | 1.65 |
| 46 | 2389 | 2607 | 1.09 |
| 47 | 3909 | 12406 | 3.17 |

Molecular weights may be characterized using high-temperature gel permeation chromatography (GPC). GPC analysis was performed using a Polymer Labs (Agilent) GPC 220 system with a refractive index detector. A Resipore column was used (300×7.5 mm). The mobile phase was 1,2,4-trichlorobenzene with a flow rate of 1 mL/min. All samples were prepared at 1 mg/mL in 1,2,4-trichlorobenzene. Loop volume was 100 μL. The system was calibrated with, and all results were comparative to, polystyrene standards with peak molecular weights of, 10110, 21810, 28770, 49170, 74800, 91800, 139400 & 230900. Standard system temperature for measurement for molecular weights of fused thiophene based polymers was 200° C.

These results demonstrate that using the reaction conditions of Table 5, mainly low molecular weight oligomers were synthesized with low yield (1-10%). For example, synthesis of Entry 45 produced about 90% monomer (i.e., n=1) and roughly 10% oligomers with n=2-3 repeat units. For Entry 46, the reaction conditions produced almost entirely monomer (i.e., about 1% oligomer) while for Entry 47, the product consisted of about 90% monomer and 10% oligomers with n=2-5 repeat units. Without being bound by theory, one reason might be due to low reactivity of FT4-H towards brominated thiophene of the DPP monomer, as seen in Example 1.

Based on the results of Example 3 provided above, thiophene-flanked FT4 as donor unit (formed in Reaction 4A) was reacted with dibromo-DPP, as shown in Reaction 4B, with the molecular weight of the product shown in Table 7.

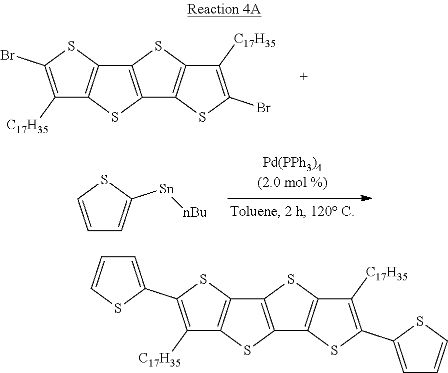

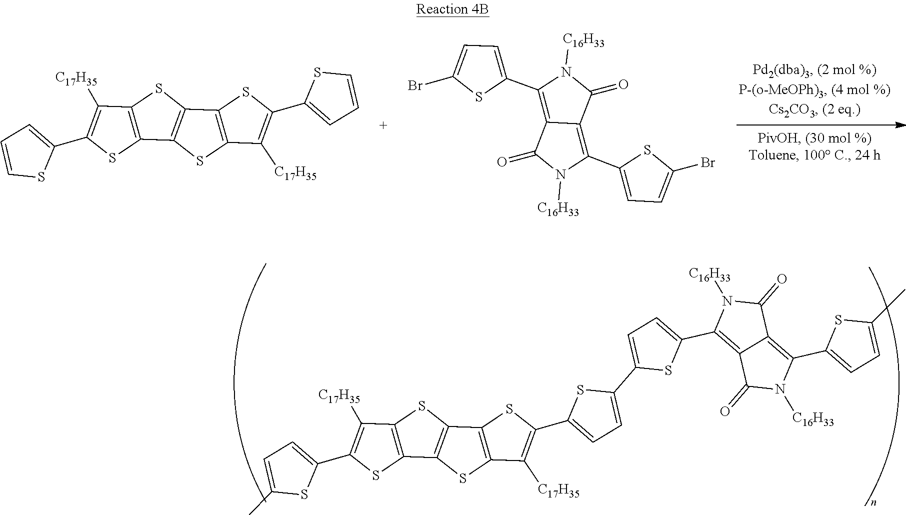

TABLE 7

| | Number | | |
|---|---|---|---|
| | Avg | | |
| | Molecular | Molecular | |
| Entry | Wt. (Mₙ) | Wt. (M_{w}) | |
| No. | (Da) | (Da) | PDI |
| 48 | 2124 | 2386 | 1.12 |

As in the reaction conditions of Table 5, Reaction 4B resulted in mainly low molecular weight oligomers (see Table 7) with low yield (1-10%).

Thus, as provided in Examples 1-3, successful synthesis of FT4-based small molecules are demonstrated and may be useful as precursor monomers for further novel OSC polymer synthesis processing, or themselves be directly utilized as organic semiconducting materials for various electronic and photonic applications (e.g., OTFT, OLED, OPV devices) or as fluorescent materials in other applications.

### Example 4—General Manufacturing Procedure for OTFT Device

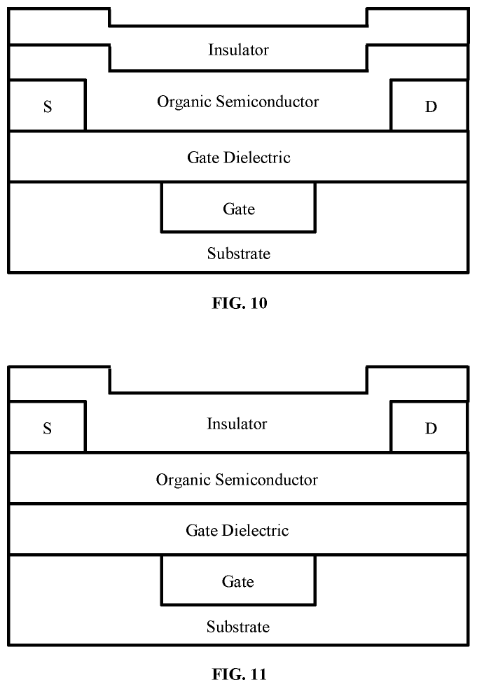

The FT4-based small molecules of Examples 1-3 may be incorporated into OTFT devices of FIGS. 10 and 11. For example, OTFT devices may be completed by forming a gate electrode over the substrate; forming a gate dielectric layer over the substrate; forming patterned source and drain electrodes over the gate dielectric layer; forming an organic semiconductor active layer over the and gate dielectric layer and forming an insulator layer over the patterned organic semiconductor active layer.

In some examples, a bottom gate, bottom contact OTFT device can be formed as following: patterning a gold (Au) or silver (Ag) gate electrode onto a substrate, followed by spin-coating a dielectric onto the substrate and treating to obtain a gate dielectric layer. After patterning Au or Ag source and drain electrodes, an OSC layer may be formed by the materials and methods as described herein to a thickness in a range of 10 nm to 200 nm. Finally, an insulator layer was positioned. One example of the formed OTFT device is shown in FIG. 10.

Thus, as presented herein, improved synthesis of FT4-based organic semiconducting small molecules by Pd-catalyzed direct (hetero)arylation for OSC layers of organic thin-film transistors are disclosed. Advantages include: (1) synthesis of novel A-D-A type conjugated small molecules with FT4 as donor unit in a one-step process with moderate to high yields, as compared with conventional C—C cross coupling reactions requiring multiple-step reactions at much higher cost; (2) using an environmentally benign direct (hetero)arylation method which avoids toxic and/or sensitive organometallic precursors used in conventional transition metal-catalyzed C—C cross coupling reactions (e.g. Suzuki and Stille coupling); (3) the novel FT4-based organic semiconducting small molecules also exhibit fluorescence properties and outstanding charge mobility for OTFT, OPV, and other organo-electronic and organo-photonic devices; and (4) compared with conventional C—C cross coupling reactions (e.g., Stille coupling), the FT4-DPP based OSC oligomers are synthesized with direct (hetero)arylation in a more environmentally benign process, without involving toxic tin precursors and byproducts.

As utilized herein, the terms “approximately,” “about,” “substantially”, and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the invention as recited in the appended claims.

As utilized herein, “optional,” “optionally,” or the like are intended to mean that the subsequently described event or circumstance can or cannot occur, and that the description includes instances where the event or circumstance occurs and instances where it does not occur. The indefinite article “a” or “an” and its corresponding definite article “the” as used herein means at least one, or one or more, unless specified otherwise.

References herein to the positions of elements (e.g., “top,” “bottom,” “above,” “below,” etc.) are merely used to describe the orientation of various elements in the FIGURES. It should be noted that the orientation of various elements may differ according to other exemplary embodiments, and that such variations are intended to be encompassed by the present disclosure.

With respect to the use of substantially any plural and/or singular terms herein, those having skill in the art can translate from the plural to the singular and/or from the singular to the plural as is appropriate to the context and/or application. The various singular/plural permutations may be expressly set forth herein for the sake of clarity.

It will be apparent to those skilled in the art that various modifications and variations can be made without departing from the spirit or scope of the claimed subject matter. Accordingly, the claimed subject matter is not to be restricted except in light of the attached claims and their equivalents.

What is claimed is:

1. A method for forming organic semiconducting materials, comprising:
providing a mixture including:
a tetrathienoacene (FT4)-based monomer, and
one of a thiophene-containing compound or an alkene-containing compound; and
reacting the FT4-based monomer with the thiophene-containing compound or the alkene-containing compound in a one-step direct arylation reaction mechanism to form a final FT4-based organic semiconducting compound.

2. The method of claim 1, wherein the reaction proceeds to a yield of at least 10% completion to form the final FT4-based organic semiconducting compound.

3. The method of claim 1, wherein the reaction proceeds to a yield of at least 20% completion to form the final FT4-based organic semiconducting compound.

4. The method of claim 1, wherein the reaction proceeds to a yield of at least 30% completion to form the final FT4-based organic semiconducting compound.

5. The method of claim 1, wherein the reaction proceeds to a yield of at least 50% completion to form the final FT4-based organic semiconducting compound.

6. The method of claim 1, wherein the final FT4-based organic semiconducting compound is an oligomer comprising at least two repeat units and at most ten repeat units.

7. The method of claim 1, wherein the final FT4-based organic semiconducting compound has a molecular weight in a range of 1000 Da to 12500 Da.

8. The method of claim 1, wherein the step of reacting is conducted in the presence of a Pd catalyst.

9. The method of claim 8, wherein the Pd catalyst comprises at least one of: Pd(OAc)₂, PdCl₂, Pd(O₂CCF₃)₂, C₈H₁₂B₂F₈N₄Pd, Pd(PPh₃)₄, Pd/C, Pd₂(dba)₃, PPh₃, P-(o-MeOPh)₃/Pd₂(dba)₃, PdCO₂(CF₃)₂, tetrakis (acetonitrile) palladium (II) tetrafluoroborate, PdCl₂(MeCN)₂, Pd₂(dba)₃.CHCl₃, Herrmann-Beller catalyst, or combinations thereof.

10. The method of claim 1, wherein the step of reacting is conducted in a solvent selected from: dimethylacetamide (DMAc), toluene, tetrahydrofuran (THF), dimethylformamide (DMF), benzotrifluoride, hexafluoroisopropanol (HFIP), 1,2-dichloroethane (DCE), dimethoxyethane (DME), hexafluorobenzene, 1,4-dioxane, mesitylene, chlorobenzene, p-xylene, o-dichlorobenzene, 1,2,4-trichlorobenzene, 1-chloronaphthalene, or combinations thereof.

11. The method of claim 1, wherein the final FT4-based organic semiconducting compound has a fluorescence intensity of at least 200 a.u., as measured 200 nm to 750 nm.

12. The method of claim 1, wherein the mixture further includes at least one of an additive, an oxidant, a base, or a ligand.

13. An organic semiconducting material selected from the group consisting of: 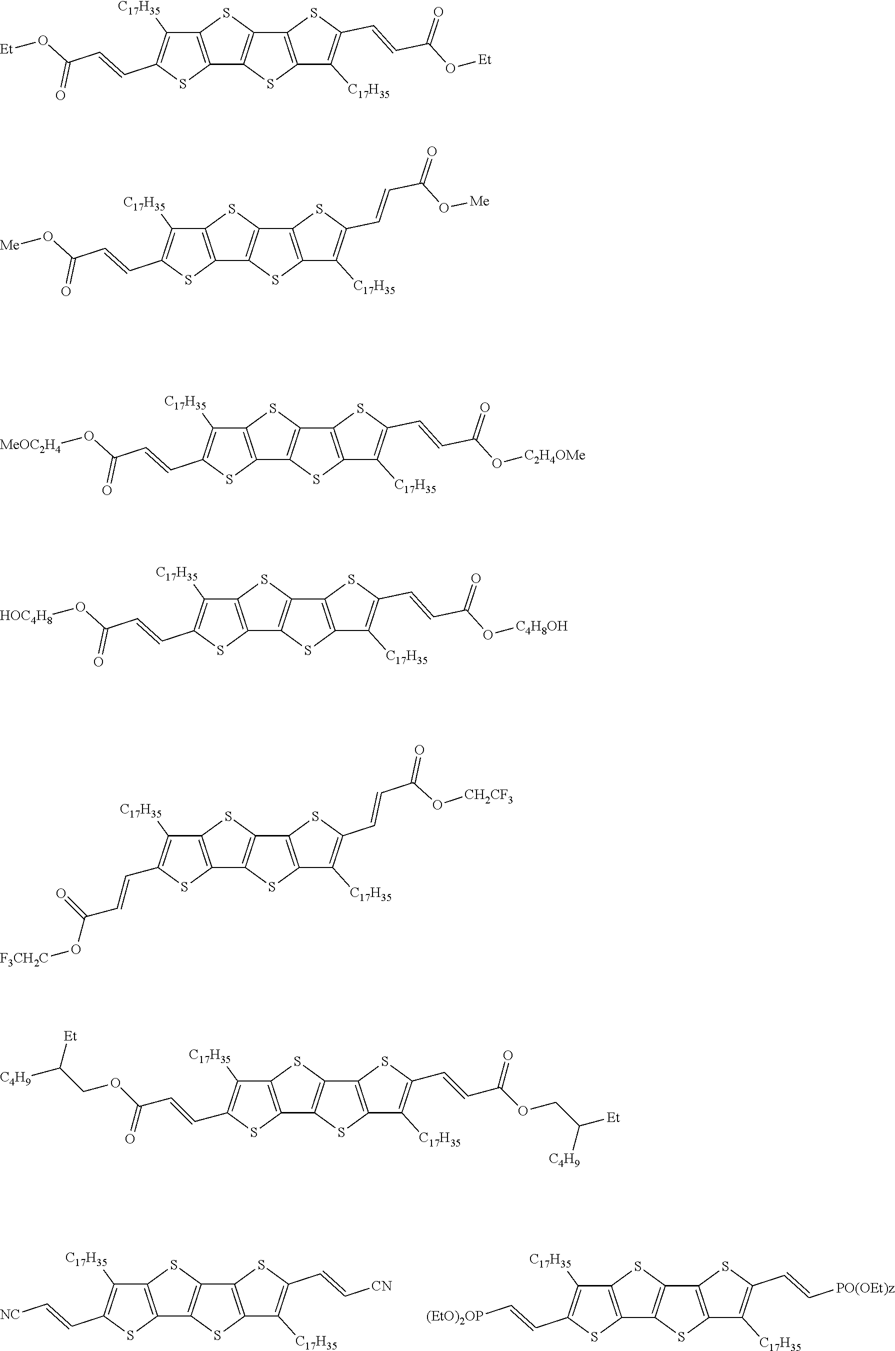 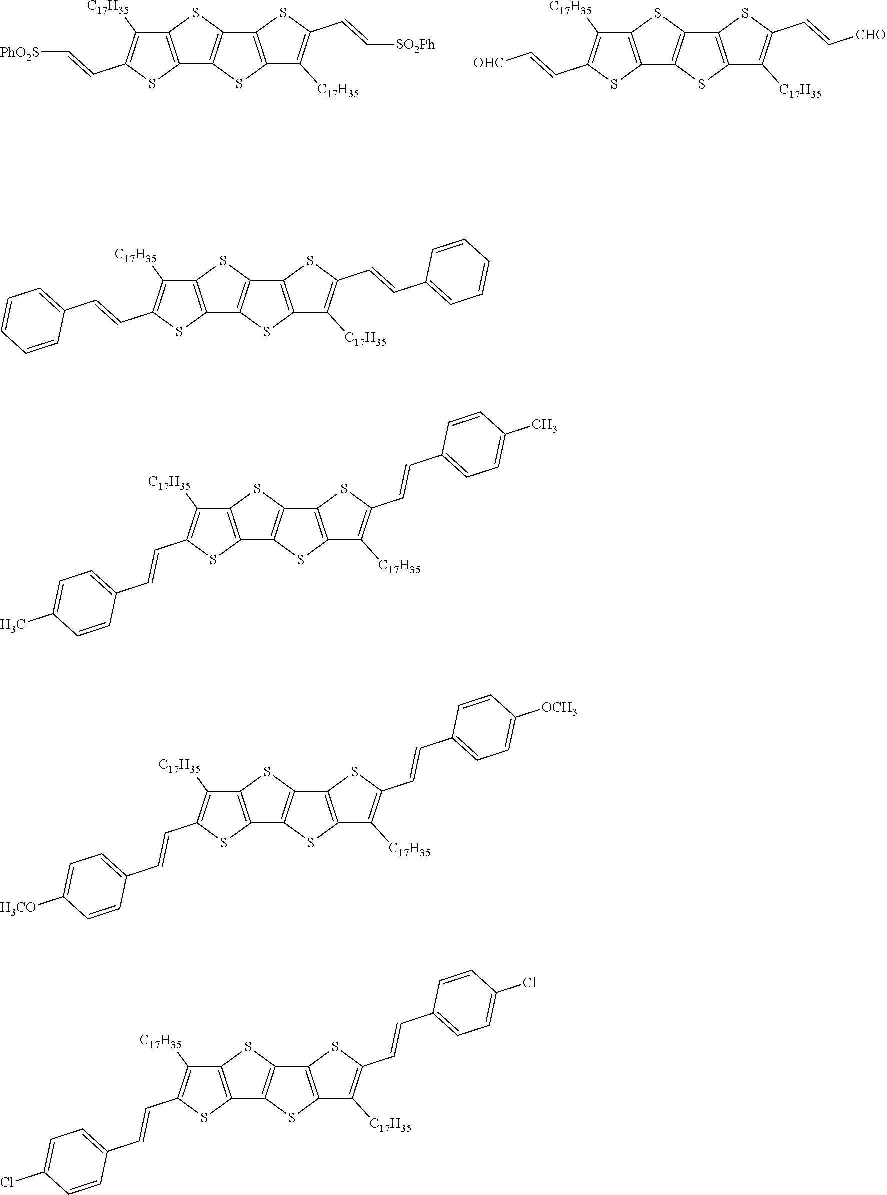 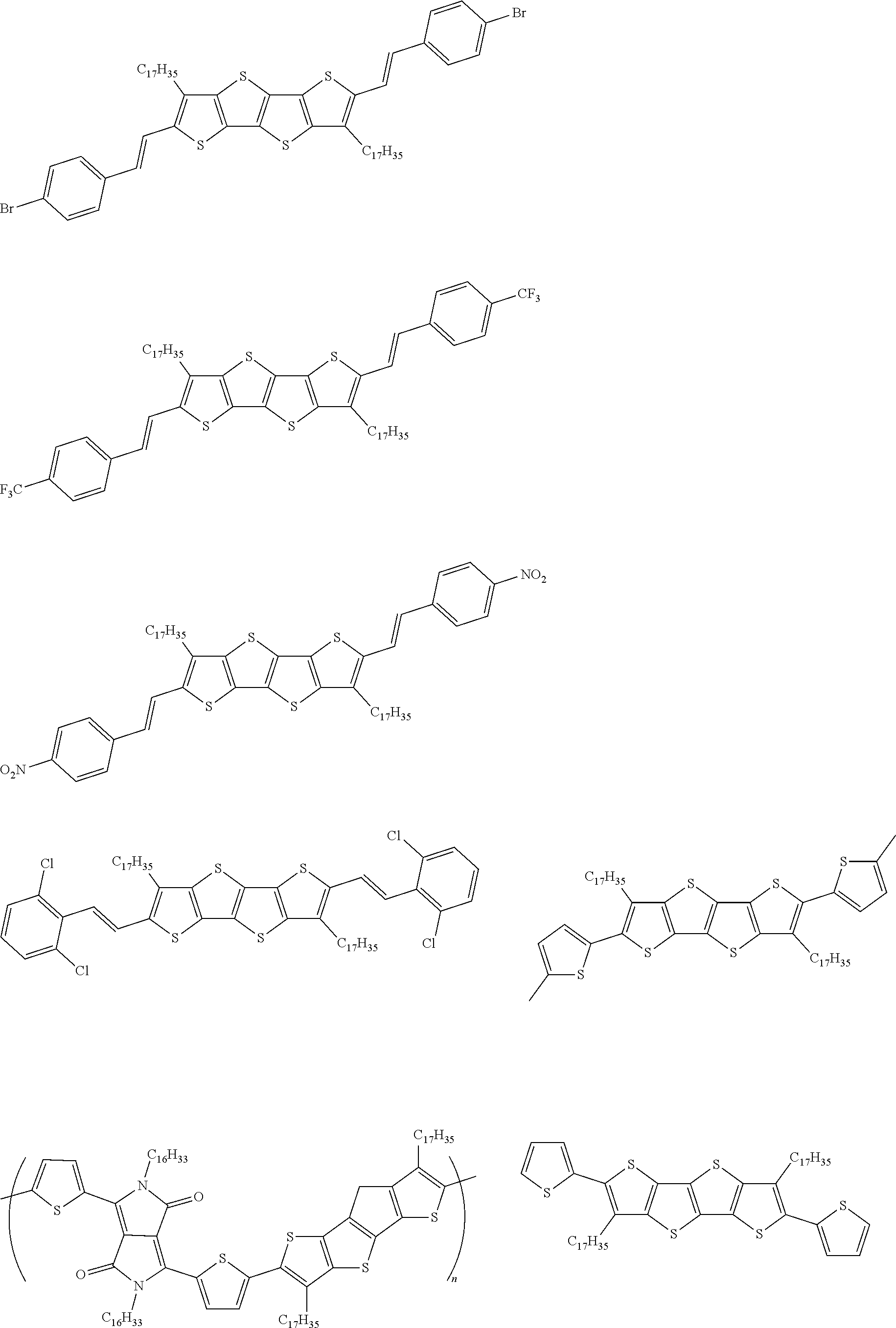 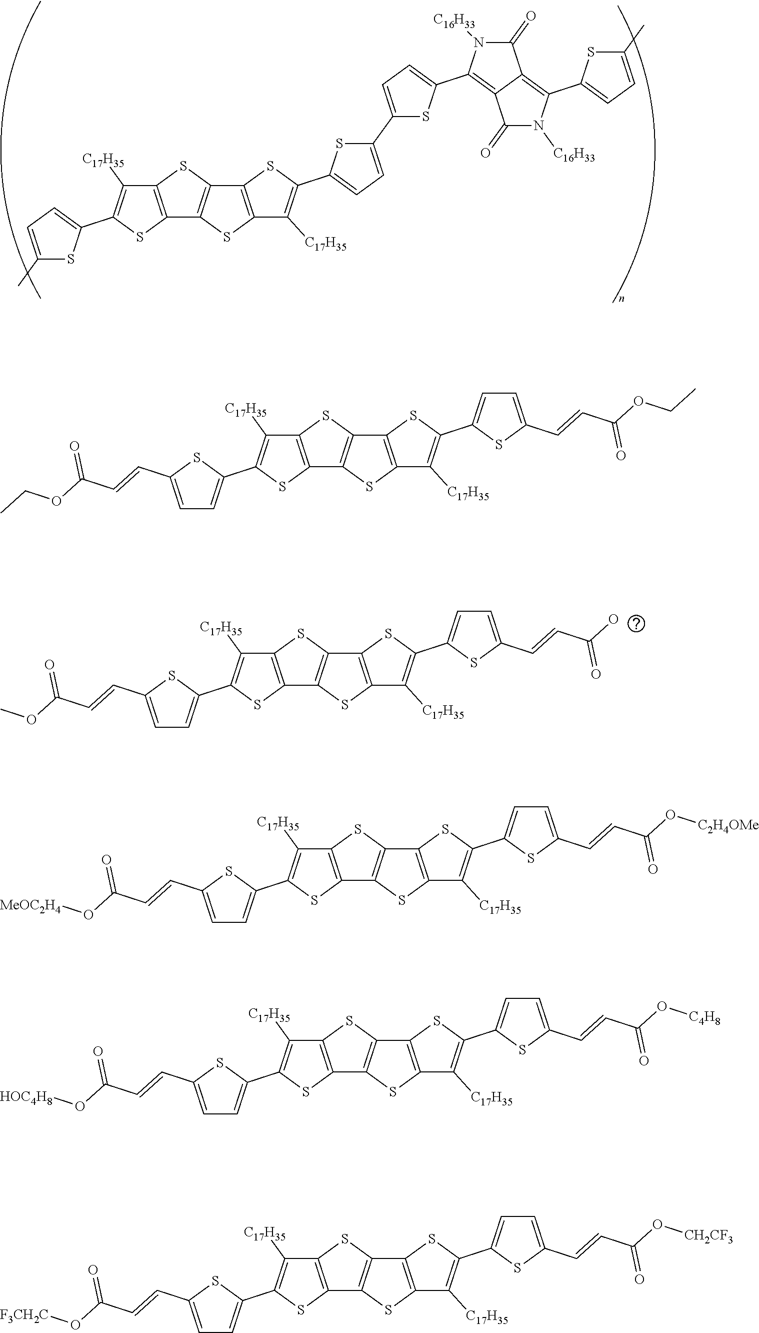 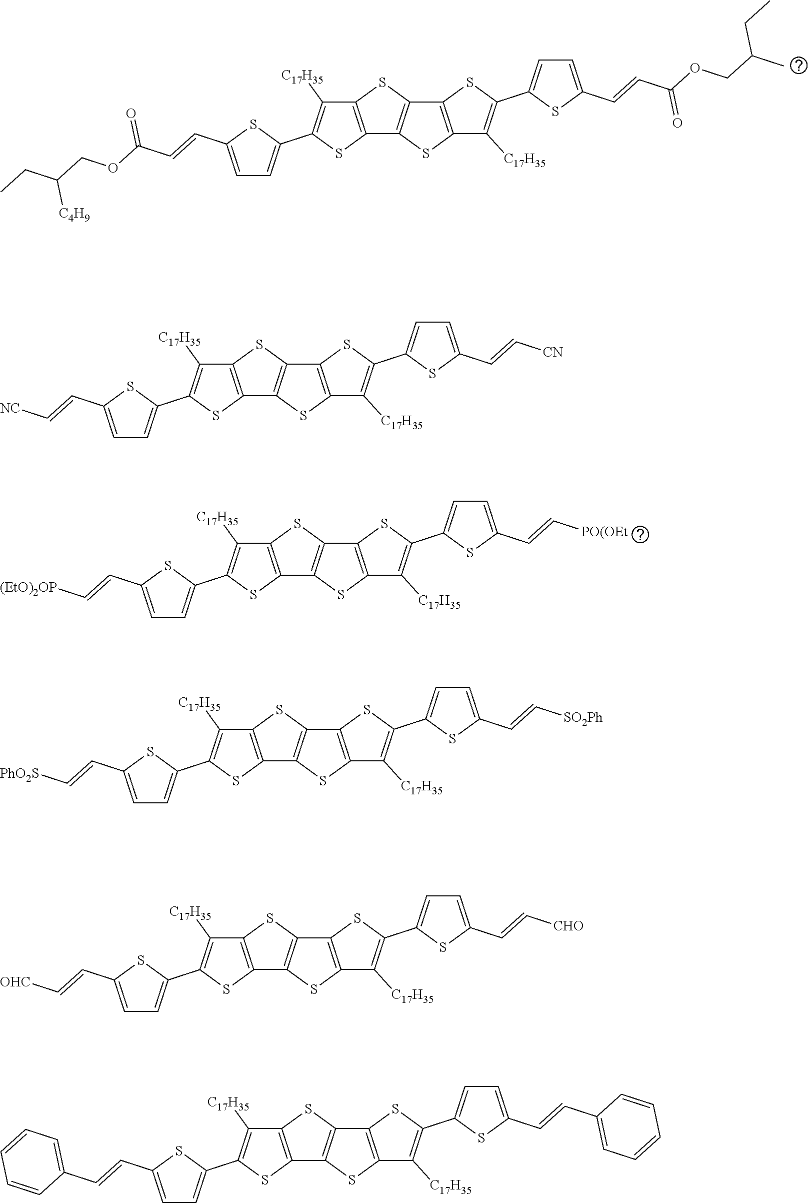 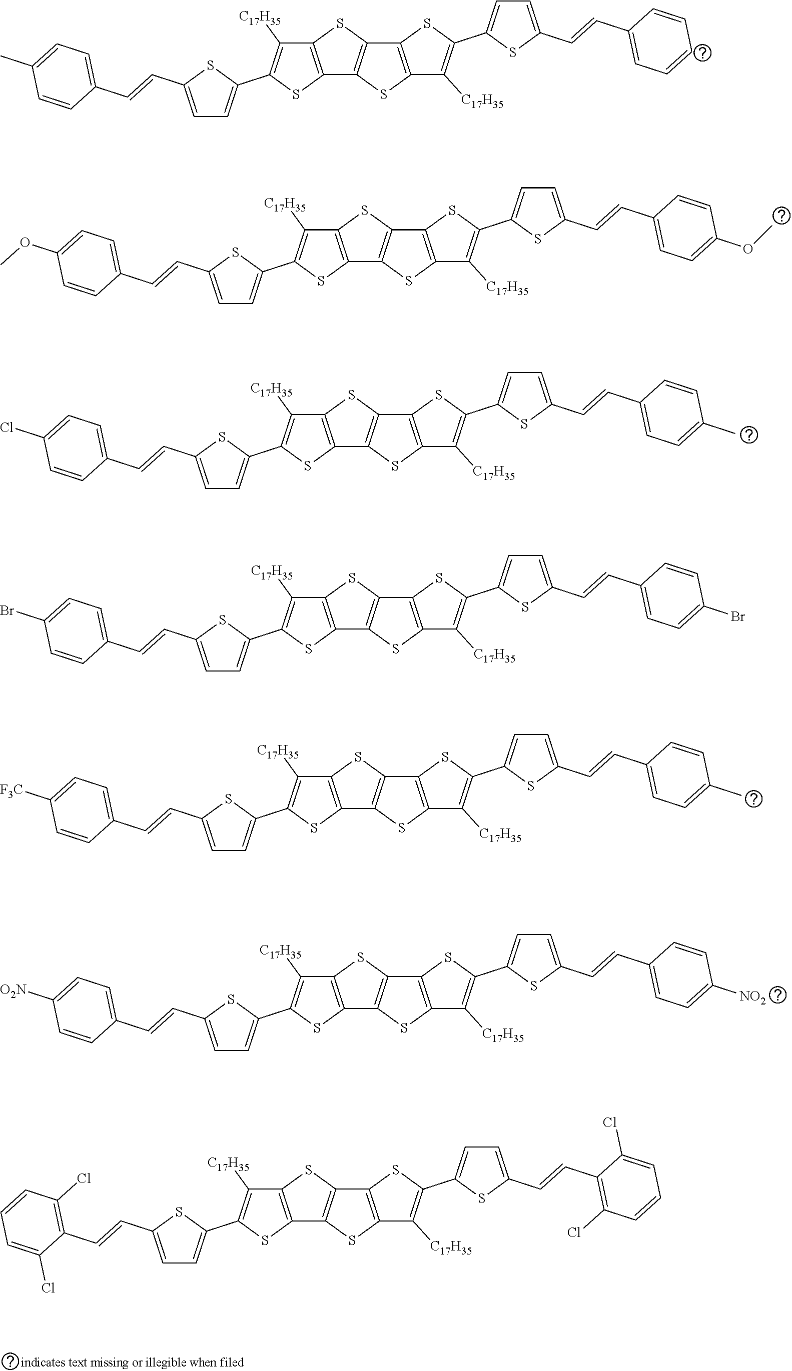
where n=2, 3, 4, or 5.
